(19) United States
(12) Patent Application Publication
(10) Pub. No.: US 2024/0398711 A1
(43) Pub. Date: Dec. 5, 2024

(54) MARIBAVIR COMPOSITIONS AND USES THEREOF

(71) Applicant: Takeda Pharmaceutical Company Limited, Osaka (JP)

(72) Inventor: Katsuhiko SUEDA, Lexington, MA (US)

(21) Appl. No.: 18/678,709

(22) Filed: May 30, 2024

Related U.S. Application Data
(60) Provisional application No. 63/633,277, filed on Apr. 12, 2024.
(60) Provisional application No. 63/525,228, filed on Jul. 6, 2023.
(60) Provisional application No. 63/470,161, filed on May 31, 2023.

(51) Int. Cl. A61K 9/16; A61K 9/00; A61K 9/10; A61K 31/7056

(52) U.S. Cl. CPC A61K 9/1652; A61K 9/0053; A61K 9/10; A61K 9/1623; A61K 9/1635; A61K 9/1664; A61K 9/1682; A61K 31/7056

(57) ABSTRACT

The present disclosure provides novel methods and compositions for using maribavir in the treatment of pediatric and adolescent patient populations. The present disclosure also provides compositions of maribavir in the form of a powder for oral suspension, which are useful for patients with difficulty swallowing pills, including pediatric and adolescent patient populations.

### CROSS-REFERENCE TO RELATED APPLICATIONS

This application claims priority to and benefit of U.S. provisional application nos. 63/470,161, filed May 31, 2023; 63/525,228, filed Jul. 6, 2023; and 63/633,277, filed Apr. 12, 2024; the entire contents of each of which is incorporated herein by reference.

### BACKGROUND

Cytomegalovirus (CMV) is a member of the herpesvirus family. Human cytomegalovirus (HCMV) infection is common, with serologic evidence of prior infection in 40% to 100% of various adult populations. However, serious HCMV disease occurs almost exclusively in individuals with compromised or immature immune systems. Cytomegalovirus remains a significant problem for patients undergoing various types of transplants that are associated with the use of potent immunosuppressive chemotherapy, including hematopoietic stem cell transplants (HSCTs) and solid organ transplants (SOTs).

Currently available systemic anti-CMV agents include intravenous (IV) ganciclovir, oral valganciclovir (a prodrug of ganciclovir with improved bioavailability), IV foscarnet, IV cidofovir, letermovir (IV and oral), and maribavir (oral). In the transplant setting, ganciclovir is the drug that has been studied most extensively, both for prevention and treatment of CMV infection or disease. While all of these drugs are effective anti-CMV agents, their use is limited by their respective toxicities, most notably bone marrow suppression caused by ganciclovir/valganciclovir, and renal impairment caused by foscarnet and cidofovir. These toxicities are of particular concern in transplant patients, in whom the bone marrow has been ablated or significantly suppressed (stem cell transplant (SCT) patients), who receive ongoing chemotherapy specifically intended to suppress their immune system to prevent organ rejection (SOT patients) or graft versus host disease (GVHD; in SCT patients), or who may require the use of other therapies that are potentially toxic to the kidneys or other organs (SOT and SCT patients). Development of antiviral resistance to currently available anti-CMV agents is also an ongoing clinical problem in SOT and HSCT patients that leads to graft loss and even mortality for some transplant patients. Letermovir is a newer agent, which targets CMV deoxyribonucleic acid (DNA) terminase; however, it is indicated for the prophylaxis of CMV reactivation and disease in adult CMV seropositive recipients [R+] of an allogeneic HSCT. Ganciclovir IV is indicated in the European Union (EU) in adults and adolescents at least 12 years of age for the treatment of CMV disease in immunocompromised patients, and maribavir is approved for use in the United States (US) in adults and pediatric patients at least 12 years of age and weighing at least 35 kg with post-transplant CMV infection or disease that is refractory to treatment (with or without genotypic resistance) with ganciclovir, valganciclovir, cidofovir, or foscarnet. There are currently no approved agents for the treatment of CMV infection or disease in pediatric patients<12 years of age and/or weighing less than 35 kg.

### SUMMARY

Maribavir is a benzimidazole riboside, and is an orally available antiviral medication against CMV. Maribavir is also known under its trade name LIVTENCITY™ Typically, patients (e.g., adults and/or pediatric patients at least 12 years of age and weighing at least 35 kg) are administered 400 mg of maribavir tablets orally, twice daily. However, in some aspects, the present disclosure recognizes that many pediatric and adolescent patients are less than 12 years of age and/or have a body weight less than 35 kg and/or may have difficulty swallowing pills. The present disclosure provides, among other things, methods and compositions for treating such patient populations.

The present disclosure further provides methods for treating CMV infection or disease in a patient less than 12 years of age, comprising administering a therapeutically effective amount of a pharmaceutical composition (e.g., an oral liquid formulation) comprising maribavir. The present disclosure further provides methods for treating CMV infection or disease in a patient less than 6 years of age, comprising administering a therapeutically effective amount of a pharmaceutical composition (e.g., an oral liquid formulation) comprising maribavir. In some embodiments, the pharmaceutical composition comprises maribavir, a sweetener, and a flavorant. In some embodiments, the present disclosure provides methods of treating CMV infection or disease in a patient suffering therefrom comprising orally administering maribavir to the patient, wherein the patient is less than 6 year of age.

In some embodiments, the present disclosure relates to a pharmaceutical composition for oral administration, where the composition contains coated particles comprising an active substance (e.g., maribavir) which has an unpleasant and/or bitter taste when orally administered. In some embodiments, the present disclosure relates to a pharmaceutical composition containing bitter taste-masking coated particles comprising maribavir. In some embodiments, such pharmaceutical compositions are in a form suitable for aqueous suspension. In some embodiments, the pharmaceutical composition is a powder, e.g., a powder for oral suspension.

### BRIEF DESCRIPTION OF THE DRAWINGS

FIG. 1 shows Flavor Profile Method definitions and scales for quantitative comparison of sensory information.

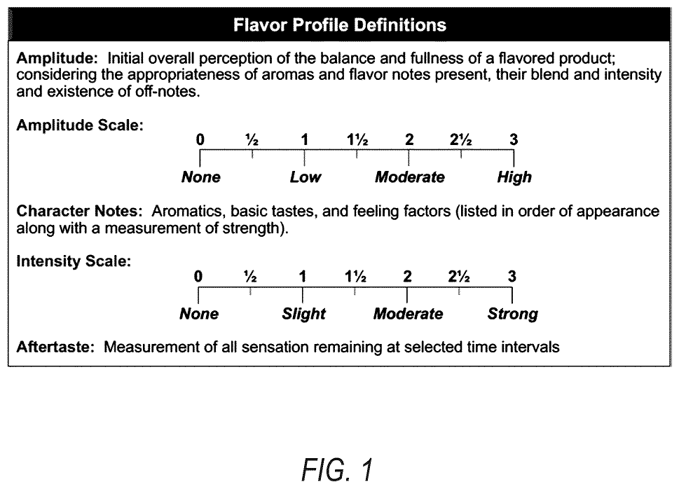

FIG. 2 shows percent dissolution of batches of formulated maribavir with different particle sizes.

### DETAILED DESCRIPTION

Maribavir ((2S,3S,4R,5S)-2-(5,6-dichloro-2-(isopropylamino)-1H-benzo[d]imidazol-1-yl)-5-(hydroxymethyl)tetrahydrofuran-3,4-diol), a compound having the chemical structure:

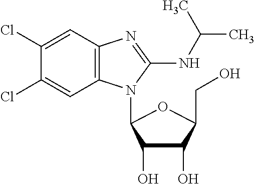

is a potent and orally bioavailable antiviral for the treatment of CMV infection or disease in transplant recipients. Pharmaceutically acceptable salts of maribavir are also within the scope of the present disclosure, e.g., in the compositions and methods described herein. Transplant recipients are at significant risk of CMV infection or disease. While maribavir is approved for treating CMV in adults and adolescents at least 12 years of age who weigh at least 35 kg, many pediatric and adolescent patients are less than 12 years of age and/or have a body weight less than 35 kg (collectively, “pediatric patients”) and many have difficulty swallowing pills. The present disclosure encompasses the recognition that novel dosing and/or formulations are needed in order to treat CMV in pediatric patients and provides such methods and formulations. The present disclosure also encompasses the recognition that novel formulations are needed for adult patients who have difficulty swallowing pills and provides such formulations.

Previous studies assessing the palatability of maribavir characterized it as having a strong and lingering bitter taste and secondary aromatic off-notes at clinically-relevant doses, posing a taste masking challenge in pediatric patients, particularly those who are too young to swallow tablets (e.g., less than 6 years of age). In order to provide a palatable formulation for pediatric use, a taste-masking strategy that minimizes the availability of maribavir in the oral cavity was explored. The taste-masking approach utilized a polymer for coating the maribavir substance, where the polymer is insoluble at neutral pH (i.e., of saliva) but soluble at acidic pH (i.e., of gastric fluid). By encapsulating maribavir inside a coating polymer to create a powder substance for liquid suspension, it was thought that maribavir would be unavailable for taste perception in the mouth, but properly released in the stomach for absorption.

In the first part (“Part 1”) of a clinical study (NCT04131556), two maribavir oral suspension formulations were tested, each containing the same coating polymer but with different amounts. The formulations were evaluated for palatability and bioavailability of maribavir relative to the tablet formulation. Based on pharmacokinetic results from Part 1 of the study, a second part of the study had been planned to test one of the two formulations for dose proportionality and impact of food on the rate and extent of absorption of the selected formulation. However, the study was terminated after Part 1 because “based on the planned interim analysis of the data of Part 1, palatability of both pediatric formulations was not acceptable.”

While not wishing to be bound by theory, the unacceptable palatability of the formulations tested in NCT04131556 is likely due to the inability of the formulations to fully mask the strong bitter taste of the active drug substance. Thus, the present disclosure includes a previously unrecognized source of a problem. The present disclosure provides, among other things, novel formulations that overcome prior failed attempts to create a palatable oral liquid formulation of maribavir. More specifically, employing a polymer coating technology similar to that of formulations previously tested, but through modifications of other aspects of the formulation, the present disclosure has unexpectedly provided compositions that improve liquid suspension formulations to an acceptable palatability. Such compositions also are characterized by a dissolution profile that is expected to provide bioavailability of the maribavir active ingredient comparable to that of maribavir tablets taken orally. Slow dissolution rates can be undesirable as they may lead to lower bioavailability and therefore require higher dosing and/or a less desirable taste profile, thereby impacting compliance and treatment efficacy.

In some embodiments, provided pharmaceutical compositions suitable for oral administration comprise maribavir, a pH-sensitive polymer coating, a sweetener, a flavorant, and one or more excipients (e.g., a diluent, disintegrant, or suspending agent), which may then be made into a liquid suspension. Such pharmaceutical compositions comprise intragranular particles having particle sizes found to be useful for optimal dissolution. Provided oral liquid formulations can be administered to patients who cannot swallow a tablet (e.g., patients less than 6 years of age, or adult patients who have difficulty swallowing pills) for the treatment of CMV.

Provided methods of the disclosure include treating CMV infection or disease in a patient suffering therefrom comprising orally administering maribavir to the patient, wherein the patient is at least 12 years of age and less than 18 years of age, and wherein the amount of maribavir administered is based on body weight (e.g., 100 mg twice daily for patients having a body weight of at least 10 kg and less than 14 kg; 200 mg twice daily for patients having a body weight of at least 14 kg and less than 25 kg; or 400 mg twice daily for patients having a body weight of at least 25 kg).

Provided methods of the disclosure also include treating CMV infection or disease in a patient suffering therefrom comprising orally administering maribavir to the patient, wherein the patient is at least 6 years of age and less than 12 years of age, and wherein the amount of maribavir administered is based on body weight (e.g., 100 mg twice daily for patients having a body weight of at least 10 kg and less than 14 kg; 200 mg twice daily for patients having a body weight of at least 14 kg and less than 25 kg; or 400 mg twice daily for patients having a body weight of at least 25 kg).

In addition, the present disclosure provides methods for treating CMV infection or disease in a patient less than 6 years of age, comprising administering a therapeutically effective amount of a pharmaceutical composition (e.g., an oral liquid formulation) comprising maribavir. In some embodiments, the pharmaceutical composition comprises maribavir, a sweetener, and a flavorant. In some embodiments, the amount of maribavir administered is based on body weight (e.g., 50 mg once daily for patients having a body weight between about 5 and 7 kg; 50 mg twice daily for patients having a body weight between about 7 and 10 kg; 100 mg twice daily for patients having a body weight between about 10 and 14 kg; 200 mg twice daily for patients having a body weight between about 14 and 25 kg; and 400 mg twice daily for patients having a body weight greater than or equal to about 25 kg).

In some embodiments, provided methods further comprise a step of confirming CMV viremia clearance in the patient. In some embodiments, CMV viremia clearance is defined as having a plasma CMV DNA concentration below a lower limit of quantification (e.g., <137 International units per milliliter [IU/mL] in 2 consecutive post baseline samples, separated by at least 5 days).

### Definitions

The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within (i.e., ±) 10%, 9%, 8%, 7%, 6%, 5%, 4%, 3%, 2%, 1%, or less of the referred value. For example, a dose that comprises “about 200 mg” of maribavir encompasses any amount of maribavir within a range of 180 mg to 220 mg. The terms “about,” “approximately,” and the symbol “˜” are used interchangeably herein and have the same meaning.

As used herein, the term “administering” or “administration” typically refers to administration of a composition to a subject to achieve delivery of an active agent to a site of interest (e.g., a target site which may, in some embodiments, be a site of disease or damage, and/or a site of responsive processes, cells, tissues, etc.) As will be understood by those skilled in the art, reading the present disclosure, in some embodiments, one or more particular routes of administration may be feasible and/or useful in the practice of the present disclosure. In some embodiments, administration may be oral. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing.

As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

The terms “treat,” “treatment,” or “treating,” as used herein, refers to partially or completely alleviating, inhibiting, ameliorating and/or relieving a disorder or condition, or one or more symptoms of the disorder or condition. In some embodiments, treatment may be administered after one or more symptoms have developed. In some embodiments, the term “treating” includes halting the progression of a disease or disorder. Treatment may also be continued after symptoms have resolved, for example to prevent or delay their recurrence. Thus, in some embodiments, the term “treating” includes preventing relapse or recurrence of a disease or disorder.

### Dosing Regimens

Provided herein are methods of treating a patient suffering from CMV, comprising administering maribavir (e.g., by administering a composition that comprises and/or delivers maribavir as described herein) to the patient. In some embodiments, provided methods comprise administering about 400 mg of maribavir orally to the patient twice daily. In some embodiments, provided methods comprise administering about 200 mg of maribavir orally to the patient twice daily. In some embodiments, provided methods comprise administering about 100 mg of maribavir orally to the patient twice daily. In some embodiments, provided methods comprise administering about 50 mg of maribavir orally to the patient twice daily. In some embodiments, provided methods comprise administering about 50 mg of maribavir orally to the patient once daily.

Maribavir can be administered with or without food. In some embodiments, maribavir is administered as 200 mg tablets. In some embodiments, maribavir is administered as 200 mg liquid suspension for oral ingestion. In some embodiments, maribavir is administered as 50 mg liquid suspension for oral ingestion.

In some embodiments, maribavir can be administered with an antacid. In some embodiments, a provided pharmaceutical composition can be administered with an antacid. In some embodiments, a provided pharmaceutical composition is administered to a patient in need thereof, wherein the patient has received or is receiving an antacid. In some embodiments, the antacid is omeprazole, esomeprazole, rabeprazole, lansoprazole, benzimidazole, pantoprazole, cimetidine, ranitidine, or famotidine. In some embodiments, maribavir is administered with rabeprazole. In some embodiments, maribavir is administered to a patient in need thereof, wherein the patient is receiving or has received rabeprazole.

In some embodiments, the amount of maribavir administered is based on patient bodyweight. In some embodiments, provided methods comprise methods of treating CMV infection or disease in a patient suffering therefrom comprising orally administering maribavir to the patient, wherein the patient is at least 12 years of age and less than 18 years of age, and wherein the amount of maribavir administered is 100 mg twice daily for patients having a body weight of at least 10 kg and less than 14 kg, 200 mg twice daily for patients having a body weight of at least 14 kg and less than 25 kg, or 400 mg twice daily for patients having a body weight of at least 25 kg. In some embodiments, provided methods comprise methods of treating CMV infection or disease in a patient suffering therefrom comprising orally administering maribavir to the patient, wherein the patient is at least 12 years of age and less than 18 years of age, and wherein the amount of maribavir administered is 300 mg twice daily for patients having a body weight of at least 10 kg and less than 14 kg, 600 mg twice daily for patients having a body weight of at least 14 kg and less than 25 kg, or 1200 mg twice daily for patients having a body weight of at least 25 kg, wherein the patient is coadministered with an anticonvulsant. In some embodiments, provided methods comprise methods of treating CMV infection or disease in a patient suffering therefrom comprising orally administering maribavir to the patient, wherein the patient is at least 12 years of age and less than 18 years of age, and wherein the amount of maribavir administered is 200 mg twice daily for patients having a body weight of at least 10 kg and less than 14 kg, 400 mg twice daily for patients having a body weight of at least 14 kg and less than 25 kg, or 800 mg twice daily for patients having a body weight of at least 25 kg, wherein the patient is coadministered with carbamazepine. In some embodiments, provided methods comprise methods of treating CMV infection or disease in a patient suffering therefrom comprising orally administering maribavir to the patient, wherein the patient is at least 12 years of age and less than 18 years of age, and wherein the amount of maribavir administered is 300 mg twice daily for patients having a body weight of at least 10 kg and less than 14 kg, 600 mg twice daily for patients having a body weight of at least 14 kg and less than 25 kg, or 1200 mg twice daily for patients having a body weight of at least 25 kg, wherein the patient is coadministered with carbamazepine. In some embodiments, provided methods comprise methods of treating CMV infection or disease in a patient suffering therefrom comprising orally administering maribavir to the patient, wherein the patient is at least 12 years of age and less than 18 years of age, and wherein the amount of maribavir administered is 300 mg twice daily for patients having a body weight of at least 10 kg and less than 14 kg, 600 mg twice daily for patients having a body weight of at least 14 kg and less than 25 kg, or 1200 mg twice daily for patients having a body weight of at least 25 kg, wherein the patient is coadministered with phenobarbital. In some embodiments, provided methods comprise methods of treating CMV infection or disease in a patient suffering therefrom comprising orally administering maribavir to the patient, wherein the patient is at least 12 years of age and less than 18 years of age, and wherein the amount of maribavir administered is 300 mg twice daily for patients having a body weight of at least 10 kg and less than 14 kg, 600 mg twice daily for patients having a body weight of at least 14 kg and less than 25 kg, or 1200 mg twice daily for patients having a body weight of at least 25 kg, wherein the patient is coadministered with phenytoin. In some embodiments, provided methods include methods of treating CMV infection or disease in a patient suffering therefrom comprising orally administering maribavir to the patient, wherein the patient is at least 6 years of age and less than 12 years of age, and wherein the amount of maribavir administered is 100 mg twice daily for patients having a body weight of at least 10 kg and less than 14 kg, 200 mg twice daily for patients having a body weight of at least 14 kg and less than 25 kg, or 400 mg twice daily for patients having a body weight of at least 25 kg. In some embodiments, provided methods include methods of treating CMV infection or disease in a patient suffering therefrom comprising orally administering maribavir to the patient, wherein the patient is at least 6 years of age and less than 12 years of age, and wherein the amount of maribavir administered is 300 mg twice daily for patients having a body weight of at least 10 kg and less than 14 kg, 600 mg twice daily for patients having a body weight of at least 14 kg and less than 25 kg, or 1200 mg twice daily for patients having a body weight of at least 25 kg, wherein the patient is coadministered with anticonvulsant. In some embodiments, provided methods include methods of treating CMV infection or disease in a patient suffering therefrom comprising orally administering maribavir to the patient, wherein the patient is at least 6 years of age and less than 12 years of age, and wherein the amount of maribavir administered is 200 mg twice daily for patients having a body weight of at least 10 kg and less than 14 kg, 400 mg twice daily for patients having a body weight of at least 14 kg and less than 25 kg, or 800 mg twice daily for patients having a body weight of at least 25 kg, wherein the patient is coadministered with carbamazepine. In some embodiments, provided methods include methods of treating CMV infection or disease in a patient suffering therefrom comprising orally administering maribavir to the patient, wherein the patient is at least 6 years of age and less than 12 years of age, and wherein the amount of maribavir administered is 300 mg twice daily for patients having a body weight of at least 10 kg and less than 14 kg, 600 mg twice daily for patients having a body weight of at least 14 kg and less than 25 kg, or 1200 mg twice daily for patients having a body weight of at least 25 kg, wherein the patient is coadministered with carbamazepine. In some embodiments, provided methods include methods of treating CMV infection or disease in a patient suffering therefrom comprising orally administering maribavir to the patient, wherein the patient is at least 6 years of age and less than 12 years of age, and wherein the amount of maribavir administered is 300 mg twice daily for patients having a body weight of at least 10 kg and less than 14 kg, 600 mg twice daily for patients having a body weight of at least 14 kg and less than 25 kg, or 1200 mg twice daily for patients having a body weight of at least 25 kg, wherein the patient is coadministered with phenobarbital. In some embodiments, provided methods include methods of treating CMV infection or disease in a patient suffering therefrom comprising orally administering maribavir to the patient, wherein the patient is at least 6 years of age and less than 12 years of age, and wherein the amount of maribavir administered is 300 mg twice daily for patients having a body weight of at least 10 kg and less than 14 kg, 600 mg twice daily for patients having a body weight of at least 14 kg and less than 25 kg, or 1200 mg twice daily for patients having a body weight of at least 25 kg, wherein the patient is coadministered with phenytoin. In some embodiments, the present disclosure provides methods of treating CMV infection or disease in a patient suffering therefrom comprising orally administering maribavir to the patient, wherein the patient is less than 6 years of age, and wherein the amount of maribavir administered is 50 mg once daily for patients having a body weight between about 5 and 7 kg, 50 mg twice daily for patients having a body weight between about 7 and 10 kg, 100 mg twice daily for patients having a body weight of at least 10 kg and less than 14 kg, 200 mg twice daily for patients having a body weight of at least 14 kg and less than 25 kg, or 400 mg twice daily for patients having a body weight of at least 25 kg. In some embodiments, the present disclosure provides methods of treating CMV infection or disease in a patient suffering therefrom comprising orally administering maribavir to the patient, wherein the patient is less than 6 years of age, and wherein the amount of maribavir administered is 150 mg once daily for patients having a body weight between about 5 and 7 kg, 150 mg twice daily for patients having a body weight between about 7 and 10 kg, 300 mg twice daily for patients having a body weight of at least 10 kg and less than 14 kg, 600 mg twice daily for patients having a body weight of at least 14 kg and less than 25 kg, or 1200 mg twice daily for patients having a body weight of at least 25 kg, wherein the patient is coadministered with anticonvulsant. In some embodiments, the present disclosure provides methods of treating CMV infection or disease in a patient suffering therefrom comprising orally administering maribavir to the patient, wherein the patient is less than 6 years of age, and wherein the amount of maribavir administered is 100 mg once daily for patients having a body weight between about 5 and 7 kg, 100 mg twice daily for patients having a body weight between about 7 and 10 kg, 200 mg twice daily for patients having a body weight of at least 10 kg and less than 14 kg, 400 mg twice daily for patients having a body weight of at least 14 kg and less than 25 kg, or 800 mg twice daily for patients having a body weight of at least 25 kg, wherein the patient is coadministered with carbamazepine. In some embodiments, the present disclosure provides methods of treating CMV infection or disease in a patient suffering therefrom comprising orally administering maribavir to the patient, wherein the patient is less than 6 years of age, and wherein the amount of maribavir administered is 150 mg once daily for patients having a body weight between about 5 and 7 kg, 150 mg twice daily for patients having a body weight between about 7 and 10 kg, 300 mg twice daily for patients having a body weight of at least 10 kg and less than 14 kg, 600 mg twice daily for patients having a body weight of at least 14 kg and less than 25 kg, or 1200 mg twice daily for patients having a body weight of at least 25 kg, wherein the patient is coadministered with carbamazepine. In some embodiments, the present disclosure provides methods of treating CMV infection or disease in a patient suffering therefrom comprising orally administering maribavir to the patient, wherein the patient is less than 6 years of age, and wherein the amount of maribavir administered is 150 mg once daily for patients having a body weight between about 5 and 7 kg, 150 mg twice daily for patients having a body weight between about 7 and 10 kg, 300 mg twice daily for patients having a body weight of at least 10 kg and less than 14 kg, 600 mg twice daily for patients having a body weight of at least 14 kg and less than 25 kg, or 1200 mg twice daily for patients having a body weight of at least 25 kg, wherein the patient is coadministered with phenobarbital. In some embodiments, the present disclosure provides methods of treating CMV infection or disease in a patient suffering therefrom comprising orally administering maribavir to the patient, wherein the patient is less than 6 years of age, and wherein the amount of maribavir administered is 150 mg once daily for patients having a body weight between about 5 and 7 kg, 150 mg twice daily for patients having a body weight between about 7 and 10 kg, 300 mg twice daily for patients having a body weight of at least 10 kg and less than 14 kg, 600 mg twice daily for patients having a body weight of at least 14 kg and less than 25 kg, or 1200 mg twice daily for patients having a body weight of at least 25 kg, wherein the patient is coadministered with phenytoin.

In some embodiments, the present disclosure provides methods of treating CMV infection or disease in a patient suffering therefrom comprising orally administering maribavir to the patient, wherein the patient is less than 12 years of age, and wherein the amount of maribavir administered is 50 mg once daily for patients having a body weight between about 5 and 7 kg, 50 mg twice daily for patients having a body weight between about 7 and 10 kg, 100 mg twice daily for patients having a body weight between about 10 and 14 kg, 150 mg twice daily for patients having a body weight between about 14 and 20 kg, 200 mg twice daily for patients having a body weight between about 20 and 26 kg, 300 mg twice daily for patients having a body weight between about 26 and 34 kg, or 400 mg twice daily for patients having a body weight greater than about 34 kg, wherein the patient is unable to or has difficulty swallowing tablets.

In some embodiments, the present disclosure provides methods of treating CMV infection or disease in a patient suffering therefrom comprising orally administering maribavir to the patient, wherein the patient is less than 6 years of age (optionally wherein the maribavir is formulated as a powder for oral suspension), and wherein the amount of maribavir administered is 50 mg once daily for patients having a body weight between about 5 and 7 kg; 50 mg twice daily for patients having a body weight between about 7 and 10 kg; 100 mg twice daily for patients having a body weight between about 10 and 14 kg; 200 mg twice daily for patients having a body weight between about 14 and 25 kg; and 400 mg twice daily for patients having a body weight greater than or equal to about 25 kg.

In some embodiments, the present disclosure provides methods of treating CMV infection or disease in a patient suffering therefrom comprising orally administering maribavir to the patient, wherein the patient is at least 6 years of age and less than 12 years of age (optionally wherein the maribavir is formulated as a powder for oral suspension), and wherein the amount of maribavir administered is 100 mg twice daily for patients having a body weight between about 10 and 14 kg; 200 mg twice daily for patients having a body weight between about 14 and 25 kg; and 400 mg twice daily for patients having a body weight greater than or equal to about 25 kg.

The skilled artisan will appreciate that dosing may be adjusted based on observed pharmacokinetics (PK) (e.g., mean and individual area under the curve (AUC), minimum concentration (Cmin), or Cmax in children compared to the target AUC, Cmin, and Cmax range based on adult data). For example, if AUC and Cmin are found to be consistently below 128 h*μg/mL and 4.9 μg/mL, in subjects with body weights such that they initially receive 200 mg BID maribavir, then the dosing regimen can be adjusted to 400 mg BID. Alternatively, if it is found that the AUC exceeds 776 h*μg/mL or Cmax exceeds 70 μg/mL in patients with body weight such that they received 400 mg BID maribavir, then the dose should be adjusted to 200 mg BID. In some embodiments, a mean AUC in accordance with provided methods ranges from 128 to 380 h*μg/mL.

In some embodiments, provided methods and compositions are for treating CMV infection or disease in patients less than 6 years of age.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side effects. Dosage amounts and intervals can be adjusted individually to provide levels of the administered compound effective for the particular clinical indication being treated. This will provide a therapeutic regimen that is commensurate with the severity of the individual's disease state.

In some embodiments, provided methods comprise administering maribavir to a patient in need thereof in a therapeutically effective amount. In some embodiments, provided methods comprise administering maribavir to a patient in need thereof, according to a regimen established to achieve one or more particular effects (e.g., a particular maximum concentration and/or a particular area under the curve and/or a particular half-life and/or a particular target exposure, e.g., for a particular fraction of time between doses) in a relevant population of patients. In some such embodiments, a relevant population of patients are those less than 6 years of age. In some embodiments, a relevant population of patients are those with a body weight less than 35 kg.

In some embodiments, a particular effect is or comprises a particular mean maximum concentration (Cmax), which can be extrapolated from studies in other patient populations, including adults. In some embodiments, a particular effect is or comprises a particular mean plasma concentration maintained over a particular period of time, which can be extrapolated from studies in other patient populations, including adults. In some embodiments, a particular effect is or comprises a particular mean Cmax and a particular mean plasma concentration maintained over a particular period of time, which can be extrapolated from studies in other patient populations, including adults. In some embodiments, a particular effect is or comprises a particular mean area under the curve over 168 hours or one week (AUC₍₀₁₆₈₎), which can be extrapolated from studies in other patient populations, including adults. In some embodiments, a particular effect is or comprises a particular half-life (t_{1/2}), which can be extrapolated from studies in other patient populations, including adults.

In some embodiments, maribavir is administered each day. In some embodiments, maribavir is administered daily for at least 2 weeks, at least 3 weeks, at least 4 weeks, at least 5 weeks, at least 6 weeks, at least 7 weeks, or at least 8 weeks.

### Patients

As described above, provided methods include treating cytomegalovirus (CMV) infection or disease in a patient suffering therefrom comprising administering maribavir to the patient, wherein the patient is less than 18 years of age. In some embodiments, a patient suffers from post-transplant CMV infection or disease. In some embodiments, a patient is a transplant recipient. In some embodiments, a patient is a hematopoietic stem cell transplant recipient. In some embodiments, a patient is a solid organ transplant recipient (e.g., liver, kidney, lung, heart, pancreas, intestine). In some embodiments, the patient does not have CMV tissue invasive disease involving the central nervous system (CNS) or retina prior to treatment with maribavir. In some embodiments, a patient administered a provided dosing regimen has tissue invasive disease.

In some embodiments, a patient administered a provided dosing regimen (i.e., for CMV) has CMV syndrome. In some embodiments, CMV syndrome is characterized by detection of CMV in blood by viral culture, antigenemia, or a DNA or ribonucleic acid-based assay together with at least 2 of the following:
a. Fever≥38° C. for at least 2 days.
b. New or increased malaise (toxicity grade≥2) or new or increased fatigue (toxicity grade≥3) (National Cancer Institute: Common Terminology Criteria for Adverse Events, version 4.0).
c. Leukopenia or neutropenia on 2 separate measurements at least 24 hours apart defined as: a white blood cell (WBC) count of <3,500 cells/μL, if the WBC count prior to the development of clinical symptoms was >4,000 cells/μL or a WBC decrease of >20%, if the WBC count prior to the development of clinical symptoms was <4,000 cells/μL. The corresponding neutrophil counts are <1,500 cells/μL or a decrease of >20% if the neutrophil count before the onset of symptoms was <1,500 cells/μL.
d. ≥5% atypical lymphocytes.
e. Thrombocytopenia defined as a platelet count of <100,000 cells/μL if the platelet count prior to the development of clinical symptoms was ≥15,000 cells/mL or a decrease of >20% if the platelet count prior to the development of clinical symptoms was <115,000 cells/mL.
f. Elevation of hepatic transaminases (alanine aminotransferase or aspartate aminotransferase) to 2× upper limit of normal (applicable to nonliver transplant recipients).

In some embodiments, a patient is less than 18 years of age. In some embodiments, a patient is at least 12 years of age and less than 18 years of age. In some embodiments, a patient is less than 12 years of age. In some embodiments, a patient is less than 6 years of age. In some embodiments, the patient is an adult patient (i.e., 18 years of age or older) who has difficulty swallowing pills.

In some embodiments, a patient's body weight is less than 35 kg. In some embodiments, a patient's body weight is at least 25 kg. In some embodiments, a patient's body weight is less than 25 kg. In some embodiments, a patient's body weight is 10-25 kg. In some embodiments, a patient's body weight is at least 5 kg.

The skilled artisan is aware of methods of diagnosing CMV infection or disease in a patient. In some embodiments, a patient suffering from CMV infection or disease has a CMV deoxyribonucleic acid (DNA) screening value of ≥1365 International Units per milliliter (IU/mL) in whole blood or ≥455 IU/mL in plasma in 2 consecutive assessments within 14 days of the first dose maribavir, separated by at least 1 day, by quantitative polymerase chain reaction (qPCR).

In some embodiments, a patient is refractory to treatment with one or more other drugs to treat CMV infection or disease. In some embodiments, a patient is refractory with genotypic resistance to treatment with one or more other drugs to treat CMV infection or disease. In some embodiments, a patient is refractory without genotypic resistance to treatment with one or more other drugs to treat CMV infection or disease. In some embodiments, a patient is refractory to treatment with one or more of ganciclovir, valganciclovir, cidofovir, or foscarnet. In some embodiments, a patient is refractory to treatment with ganciclovir or valganciclovir. In some embodiments, a patient is refractory to treatment with ganciclovir. In some embodiments, a patient is refractory to treatment with valganciclovir.

In some embodiments, patients are selected based on one or more inclusion criteria described in Example 1 or Example 2.

In some embodiments, the methods of treatment disclosed herein exclude the treatment of patients who meet one or more exclusion criteria described in Example 1 or Example 2.

In some embodiments, present methods include administering maribavir and a coadministered drug (e.g., a CYP3A4 inducer such as carbamazepine, phenytoin, or phenobarbital, a p-glycoprotein inducer (P-gp), an immunosuppressant, an antacid, an antiarrhythmic such as Digoxin, ganciclovir, or valganciclovir) to a patient in need thereof.

In some embodiments, a patient is receiving or has received a cytochrome P450 3A4 (CYP3A4) inducer prior to administration of maribavir, and a provided method comprises the step of discontinuing administration of the CYP3A4 inducer prior to administration of maribavir. In some embodiments, a patient is receiving or has received a cytochrome P450 3A4 (CYP3A4) inducer, and a provided method comprises the step of increasing the amount of maribavir administered to the patient. In some embodiments, a CYP3A4 inducer is a strong CYP3A4 inducer. In some embodiments, a CYP3A4 inducer is selected from the group consisting of rifampin, avasimibe, carbamazepine, phenytoin, rifabutin, phenobarbital, and St. John's wort. In some embodiments, a CYP3A4 inducer is selected from the group consisting of rifampin, rifabutin, and St. John's wort. In some embodiments, a CYP3A4 inducer is selected from the group consisting of carbamazepine, phenytoin, and phenobarbital.

In some embodiments, a patient is receiving or has received an immunosuppressant. In some embodiments, a provided method comprises the step of monitoring the levels of immunosuppressant after commencing administration of maribavir (e.g., with comparison to a reference or standard level). In some embodiments, a provided method further comprises the step of reducing the amount of the immunosuppressant administered to the patient. In some embodiments, a provided method further comprises the step of monitoring the levels of immunosuppressant after discontinuing administration of maribavir (e.g., with comparison to a reference or standard level). In some embodiments, a provided method further comprises the step of increasing the amount of immunosuppressant administered to the patient (e.g., to the amount of immunosuppressant that was administered prior to commencing administration of maribavir). In some embodiments, an immunosuppressant is selected from the group consisting of tacrolimus, cyclosporine, everolimus, and sirolimus.

In some embodiments, a patient is receiving or has received digoxin. In some embodiments, a provided method further comprises the step of monitoring the levels of digoxin. In some embodiments, a provided method further comprises the step of reducing the amount of the digoxin administered to the patient.

In some embodiments, a patient is receiving or has received an antacid. In some embodiments, the antacid is omeprazole, esomeprazole, rabeprazole, lansoprazole, benzimidazole, pantoprazole, cimetidine, ranitidine, or famotidine. In some embodiments, the antacid is rabeprazole. In some embodiments, a patient is receiving or has received an anticonvulsant. In some embodiments, the anticonvulsant is carbamazepine. In some embodiments, the anticonvulsant is phenobarbital. In some embodiments, the anticonvulsant is phenytoin.

### Pharmaceutical Compositions Suitable for Liquid Oral Formulations

As described above, the present disclosure provides novel formulations that are suitable for liquid oral formulations of maribavir. In some embodiments, provided liquid suspension formulations sufficiently reduce the bitter taste of maribavir. To successfully achieve taste masking of the bitter maribavir flavor, provided formulations employ both polymer coated maribavir granules and other agents (e.g., sweetener and flavorant). Such formulations can be administered to patients who are unable to swallow tablet formulations of maribavir. In some embodiments, a provided oral pharmaceutical composition comprising maribavir is suitable for administration to patients less than 6 years of age.

In some embodiments, a liquid formulation for oral administration, which comprises coated, e.g., taste-masked, particles comprising at least one pharmaceutically active substance, and at least one pharmaceutically acceptable excipient, which is prepared by mixing a dry preparation to a liquid vehicle prior to oral administration, thus forming a suspension.

In some embodiments, a provided pharmaceutical composition comprises an active pharmaceutical ingredient, a diluent, and a disintegrant that are granulated into an intragranular composition (e.g., particle). In some embodiments, an intragranular particle comprises an active pharmaceutical ingredient (e.g., maribavir), a diluent, and a disintegrant. In some embodiments, an intragranular particle comprises an active pharmaceutical ingredient (e.g., maribavir), microcrystalline cellulose, and crospovidone. In some embodiments, an intragranular composition is prepared by granulation (e.g., by fluidized rotor granulator) of an active pharmaceutical ingredient (e.g., maribavir), a diluent, and a disintegrant. In some embodiments, an intragranular composition is prepared by granulation (e.g., by fluidized rotor granulator) of an active pharmaceutical ingredient (e.g., maribavir), microcrystalline cellulose, and crospovidone.

In some embodiments, a provided pharmaceutical composition comprises maribavir in a composition comprising other excipients (e.g., croscarmellose sodium, sodium starch glycolate, microcrystalline cellulose and crospovidone). In some embodiments, provided pharmaceutical compositions include granules comprising maribavir, microcrystalline cellulose, and crospovidone.

In some embodiments, a provided pharmaceutical composition comprises a coating enveloping the maribavir-containing components of the composition. In some embodiments, a provided pharmaceutical composition comprises a coating enveloping the maribavir-containing components, distributed within an extragranular composition. In some embodiments, a provided pharmaceutical composition comprises a mixture of coated intragranules within a matrix of extragranular components.

In some embodiments, a provided pharmaceutical composition comprises:
(i) an intragranular composition comprising maribavir, a diluent, and a disintegrant;
(ii) an enteric coating of the intragranular composition comprising a selectively soluble polymer; and
(iii) an extragranular composition comprising a diluent, a suspending agent, a sweetener, and flavorant.

In some embodiments, a provided pharmaceutical composition comprises:
(i) an intragranular composition comprising maribavir, microcrystalline cellulose, and a disintegrant;
(ii) an enteric coating of the intragranular composition comprising a selectively soluble polymer; and
(iii) an extragranular composition comprising a diluent, a suspending agent, a sweetener, and flavorant.

In some embodiments, a provided pharmaceutical composition comprises:
(i) an intragranular composition comprising maribavir, microcrystalline cellulose, and crospovidone;
(ii) an enteric coating of the intragranular composition comprising a selectively soluble polymer; and
(iii) an extragranular composition comprising a diluent, a suspending agent, a sweetener, and flavorant.

In some embodiments, a provided pharmaceutical composition comprises:
(i) an intragranular composition comprising maribavir, a diluent, and crospovidone;
(ii) an enteric coating of the intragranular composition comprising a selectively soluble polymer; and
(iii) an extragranular composition comprising a diluent, a suspending agent, a sweetener, and flavorant.

In some embodiments, a provided pharmaceutical composition comprises:
(i) an intragranular composition comprising maribavir, a diluent, and a disintegrant;
(ii) an enteric coating of the intragranular composition comprising a selectively soluble polymer; and
(iii) an extragranular composition comprising mannitol, a suspending agent, a sweetener, and flavorant.

In some embodiments, a provided pharmaceutical composition comprises:
(i) an intragranular composition comprising maribavir, microcrystalline cellulose, and crospovidone;
(ii) an enteric coating of the intragranular composition comprising a selectively soluble polymer; and
(iii) an extragranular composition comprising mannitol, a suspending agent, a sweetener, and flavorant.

In some embodiments, a provided pharmaceutical composition comprises:
(i) an intragranular composition comprising maribavir, microcrystalline cellulose, and crospovidone;
(ii) an enteric coating of the intragranular composition comprising a selectively soluble polymer; and
(iii) an extragranular composition comprising mannitol, a suspending agent, a sucralose, and flavorant.

In some embodiments, a provided pharmaceutical composition comprises:
(i) an intragranular composition comprising maribavir, microcrystalline cellulose, and crospovidone;
(ii) an enteric coating of the intragranular composition comprising a selectively soluble polymer; and
(iii) an extragranular composition comprising mannitol, microcrystalline cellulose and carboxymethylcellulose sodium, a sweetener, and flavorant.

In some embodiments, a provided pharmaceutical composition comprises:
(i) an intragranular composition comprising maribavir, microcrystalline cellulose, and crospovidone;
(ii) an enteric coating of the intragranular composition comprising a selectively soluble polymer; and
(iii) an extragranular composition comprising mannitol, microcrystalline cellulose and carboxymethylcellulose sodium, sucralose, and flavorant.

In some embodiments, a provided pharmaceutical composition comprises:
(i) an intragranular composition comprising maribavir and at least one excipient;
(ii) an enteric coating of the intragranular composition comprising a selectively soluble polymer; and
(iii) an extragranular composition comprising (a) a diluent, (b) at least one sweetener or flavorant, and (c) an other excipient;
wherein, median particle diameter of the intragranular composition is between about 100 and 400 microns, wherein median particle diameter of the coated intragranular composition is less than 500 microns, wherein dv50 (i.e., 50th percentile) particle size of the diluent is between about 30 and 180 microns.

In some embodiments, a provided pharmaceutical composition comprises:
(i) an intragranular composition comprising maribavir and at least one excipient;
(ii) an enteric coating of the intragranular composition comprising a selectively soluble polymer; and
(iii) an extragranular composition comprising (a) a mannitol, (b) at least one sweetener or flavorant, and (c) an other excipient;
wherein, median particle diameter of the intragranular composition is between about 100 and 400 microns, wherein median particle diameter of the coated intragranular composition is less than 500 microns, wherein dv50 (i.e., 50th percentile) particle size of the mannitol is between about 30 and 180 microns.

In some embodiments, a provided pharmaceutical composition comprises maribavir, a sweetener, and a flavorant. In some embodiments, a sweetener is a high intensity sweetener. In some embodiments, a sweetener is saccharin (e.g., sodium saccharin), aspartame, acesulfame potassium (Ace-K), sucralose, neotame, advantame, or a combination thereof. In some embodiments, a sweetener is or comprises a steviol glycoside or an extract of Swingle (monk) fruit. In some embodiments, a sweetener is an enoxolone glycoside. In some embodiments, a sweetener is glycyrrhizinic acid or pharmaceutically acceptable salt thereof. In some embodiments, a sweetener is sucralose.

In some embodiments, a provided pharmaceutical composition comprises maribavir, a sweetener, and a flavorant. In some embodiments, a flavorant is a pediatric-friendly flavor theme (e.g., fruit flavors).

As used herein, the term “Flavor Profile Method” is a standard of quantitative sensory information evaluation. Flavors are evaluated in terms of five major components: character attributes, attribute intensity, order of attribute appearance, aftertaste, and amplitude (the overall impression of the analyzable and non-analyzable flavor components). See CAUL, J. F. 1957. The profile method of flavor analysis. In “Advances in Food Research” ed. Mrak, E. M. and Stewart, G. F. Vol. 7, pp 1-40. Academic Press, New York, NY; and KEANE, P. 1992. The flavor profile. In ASTM Manual on Descriptive Analysis Testing” (Manual 13) ed. Hootman, R. C. pp 5-14. ASTM, Philadelphia, PA.

Several different type of coatings (e.g., enteric coatings comprising a selectively soluble polymer) can be applied individually or in combination to the overall dosage form or particles, granules or beads that make up the dosage form. A functional coating, such as an enteric polymer, may be used, to prevent or retard dissolution until the dosage form leaves the stomach. Exemplary enteric coating materials include HPMCAS, HPMCP, CAP, CAT, carboxymethylethyl cellulose, carboxylic acid-functionalized polymethacrylates, and carboxylic acid-functionalized polyacrylates. Alternatively, a “non-functional” coating, such as a sugar-containing coating to facilitate swallowing, which does not substantially affect dissolution or other pharmacokinetic properties, may be used. In some embodiments, maribavir granules are coated with a polymer that is insoluble at neutral pH (e.g., that of saliva and the oral cavity) but soluble at acidic pH (e.g., gastric pH). In some embodiments, a coating polymer is an amino methacrylate copolymer (e.g., basic butylated methacrylate copolymer), Kollicoat Smartseal, methyl methacrylate, diethylaminoethyl methacrylate copolymer, or a combination thereof. In some embodiments, a coating polymer is an amino methacrylate copolymer. In some embodiments, a coating polymer is a basic butylated methacrylate copolymer. In some embodiments, a coating polymer is a butyl methylacrylate, dimethylaminoethyl methacrylate, methyl methacrylate polymer or combination thereof, e.g., a butyl methylacrylate, dimethylaminoethyl methacrylate, methyl methacrylate copolymer. In some embodiments, a coating polymer is Eudragit® E PO (e.g., Eudragit® E PO ReadyMix, i.e., sodium lauryl sulfate, stearic acid, talc, silicon dioxide) (Eudragit® CAS Reg. No. 24938-16-7).

### Coated Intragranular Composition

In some embodiments, a provided pharmaceutical composition for oral liquid administration comprises granulated components (e.g., an intragranular composition within an enteric coating, provided within a composition of extragranular components) derived from smaller micronized excipients and active pharmaceutical ingredients. In some embodiments, for example, a pharmaceutical composition for oral liquid administration comprises maribavir with other excipients suitable for a liquid suspension formulation for oral administration. In some embodiments, maribavir, a disintegrant, and a diluent are granulated into an intragranule, which is further enveloped in a polymer coating that is insoluble at neutral pH but is soluble at acidic pH (e.g., an enteric coating comprising a selectively soluble polymer).

In some embodiments, a provided pharmaceutical composition comprises layers of components. In some embodiments, a layered pharmaceutical composition comprises radially graduated layers. In some embodiments, a layered pharmaceutical composition comprises a maribavir layer and a coating layer. In some embodiments, a layered pharmaceutical composition comprises two or more layers. In some embodiments, a pharmaceutical composition layer is or comprises a pharmaceutically active ingredient, a diluent, and a disintegrant. In some embodiments, a pharmaceutical composition layer is a pH-sensitive polymer or copolymer. In some embodiments, the pH-sensitive polymer or copolymer intermediate layer is not soluble at or about pH 7. In some embodiments, the pH-sensitive polymer or copolymer intermediate layer is soluble at or below about pH 5. In some embodiments, a provided pharmaceutical composition suitable for suspension in liquid for oral administration comprises an intragranular layer and a pH-sensitive polymer coating enveloping the intragranular layer. In some embodiments, a provided pharmaceutical composition suitable for suspension in liquid for oral administration comprises an intragranular layer and a pH-sensitive polymer or co-polymer coating, wherein the coated intragranule (e.g., an intragranule within Eudragit®) is dispersed in an extragranular composition of excipients.

In some embodiments, a provided pharmaceutical composition is or comprises granules. In some embodiments, a provided pharmaceutical composition is or comprises multi-layered granules. In some embodiments, a provided pharmaceutical composition comprises selectively soluble layers (e.g., a pH-sensitive polymeric or co-polymeric layer). In some embodiments, a provided pharmaceutical composition is or comprises layered granules further comprising an active pharmaceutical ingredient within a pH-sensitive polymer or co-polymer. In some embodiments, a layered pharmaceutical composition comprises maribavir within a Eudragit® E PO coating.

In some embodiments, a provided pharmaceutical composition can be produced by a method of coating an intragranular composition comprising maribavir with a coating layer comprising a pH-sensitive polymer or copolymer. In some embodiments, a provided pharmaceutical composition is a core granule comprising maribavir, microcrystalline cellulose, and crospovidone wherein the granule is enveloped by an enteric polymer layer (e.g., a selectively soluble polymer such as Eudragit®).

In some embodiments, a provided pharmaceutical composition comprises intragranular particles comprising an active pharmaceutical ingredient (e.g., maribavir), a diluent (e.g., microcrystalline cellulose), and a disintegrant (e.g., crospovidone). In some embodiments, a provided pharmaceutical composition comprises intragranular particles comprising an active pharmaceutical ingredient (e.g., maribavir), a diluent (e.g., microcrystalline cellulose), a disintegrant (e.g., crospovidone), and a coating comprising a pH-sensitive layer (e.g., Eudragit® E PO ReadyMix).

In some embodiments, a provided pharmaceutical composition comprises a diluent. In some embodiments, diluents can be used individually or in combination to the overall dosage form or particles, granules, or beads that make up the dosage form. In some embodiments, a diluent is selected from the group consisting of lactose, dextrin, glucose, sucrose, sorbitol, mannitol, xylitol, lactitol, isomalt, maltitol, microcrystalline cellulose, carboxymethylcellulose sodium, hydrogenated starch hydrolysates, calcium silicate, sodium silicate, potassium silicate, magnesium chloride, sodium chloride, potassium chloride, silicon dioxide, or a combination thereof. In some embodiments, a diluent is mannitol. In some embodiments, a diluent is microcrystalline cellulose.

In some embodiments, a provided pharmaceutical composition comprises a glidant. In some embodiments, a provide pharmaceutical glidant is selected from the group consisting of ascorbyl palmitate, calcium palmitate, stearic acid, magnesium stearate, colloidal silica, starch, talc, lactose, dextrin, glucose, sucrose, sorbitol, mannitol, xylitol, lactitol, isomalt, maltitol, hydrogenated starch hydrolysates, calcium silicate, sodium silicate, potassium silicate, magnesium chloride, sodium chloride, potassium chloride, or silicon dioxide.

In some embodiments, a provided pharmaceutical composition comprises mannitol.

### Intragranular Composition

Without wishing to be bound by a particular theory, it is known in the art that particle size within a composition of particles are commonly described by range distributions at the composition's 10th, 50th, and 90th percentiles. In this disclosure and to those skilled in the art, particle size distribution is a measurement that describes the variation of particles in a pharmaceutical composition according to their size. In some embodiments, an expression of a composition's distribution of sized particles may assume the particles are spherical or spherical-like in shape.

Without wishing to be bound by a particular theory, Applicant has observed that particle sizes of active pharmaceutical ingredients and/or excipients can have an effect on dissolution and therefore bioavailability of the active pharmaceutical ingredient. Therefore, one aspect of the present disclosure is the recognition that it can be advantageous to control such particle sizes in such as a manner as to effect the bioavailability, among other pharmacokinetic parameters, of the active pharmaceutical ingredient (e.g., maribavir) in a patient.

### Maribavir Drug Substance

Maribavir ((2S,3S,4R,5S)-2-(5,6-dichloro-2-(isopropylamino)-1H-benzo[d]imidazol-1-yl)-5-(hydroxymethyl)tetrahydrofuran-3,4-diol), a compound having the chemical structure:

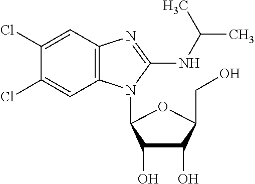

is a potent and orally bioavailable antiviral for the treatment of CMV infection or disease in transplant recipients.

In some embodiments, compositions of the present disclosure comprise a particular polymorphic form of maribavir. In some embodiments, compositions of the present disclosure comprise Form VI of maribavir (e.g., at least 90%, 95%, or 99% by weight of Form VI of maribavir), as described in U.S. Pat. No. 6,482,939. In some embodiments, compositions of the present disclosure comprise maribavir Form VI substantially free of other polymorphic forms of maribavir. As used herein, the term “polymorphic forms” includes solvates and hydrates. In some embodiments, provided compositions comprises Form VI of maribavir, as described in described in U.S. Pat. No. 6,482,939 (e.g., at least 90%, 95%, or 99% by weight of Form VI of maribavir). In some embodiments, provided compositions comprises Form VI of maribavir (e.g., at least 90%, 95%, or 99% by weight of Form VI of maribavir), as described in described in U.S. Pat. No. 6,482,939, and another polymorphic form of maribavir, e.g., as described in U.S. Pat. Nos. 6,482,939, 8,546,344, or U.S. Pat. No. 11,130,777. In some embodiments, provided compositions comprise Form VI of maribavir, as described in U.S. Pat. No. 6,482,939, and an isopropyl acetate solvate of maribavir (e.g., as described in U.S. Pat. Nos. 6,482,939, 8,546,344, or U.S. Pat. No. 11,130,777). In some embodiments, provided compositions comprise Form VI of maribavir, and less than 10%, 5%, 1%, or 0.5% by weight of another polymorphic form of maribavir, e.g., as described in U.S. Pat. Nos. 6,482,939, 8,546,344, or U.S. Pat. No. 11,130,777. In some embodiments, provided compositions comprise Form VI of maribavir, and less than 10%, 5%, 1%, or 0.5% by weight of another polymorphic form of maribavir, e.g., as described in U.S. Pat. Nos. 6,482,939, 8,546,344, or U.S. Pat. No. 11,130,777. In some embodiments, provided compositions comprise Form VI of maribavir substantially free of other polymorphic forms, e.g., as described in U.S. Pat. Nos. 6,482,939, 8,546,344, or U.S. Pat. No. 11,130,777. In some embodiments, provided compositions comprise Form VI of maribavir which is synthesized and/or crystalized by methods described in the international application PCT/US23/34933. In some embodiments, provided compositions comprise Form VI of maribavir which is crystalized by using isopropyl acetate/toluene as a crystallization solvent. In some embodiments, provided compositions comprise Form VI of maribavir which is crystalized by using maribavir seed crystals having a PSD of d (50) between about 1.00 μm and about 10.00 μm. In some embodiments, provided compositions comprise Form VI of maribavir which is crystalized by using maribavir seed crystals having a PSD of d (50) between about 1.25 μm and about 9.00 μm. In some embodiments, provided compositions comprise Form VI of maribavir which is crystalized by using maribavir seed crystals having a PSD of d (50) between about 1.75 μm and about 8.00 μm. In some embodiments, provided compositions comprise Form VI of maribavir, less than 0.05% of synthetic intermediate impurities (e.g., Compound 2, 3, and/or 4 as described in the international application PCT/US23/34933), less than 0.05% of enantiomeric impurities (e.g., D-maribavir as described in the international application PCT/US23/34933), and/or less than 0.05% of unspecified impurities as measured by HPLC.

### Particle Size

In some embodiments, a provided pharmaceutical composition comprises granules characterized by particle size (e.g., expressed as mean diameter of, for example, irregular spheroid particle), expressed as averages, percentiles, and ranges of a composition of particles (e.g., intragranular particles, coated intragranular particles, or other components of a provided pharmaceutical composition). In some embodiments, regular and irregular spheroid particles have a mean diameter and size distribution as measured by laser diffraction. Other means of measuring particle size are known and include flow imaging microscopy, X-ray powder diffraction (XRD), thermogravimetric analysis (TGA), scanning electron microscopy (SEM), dynamic vapor sorption (DVS), sieving, dynamic light scattering, etc.

### Intragranular Composition Particle Size

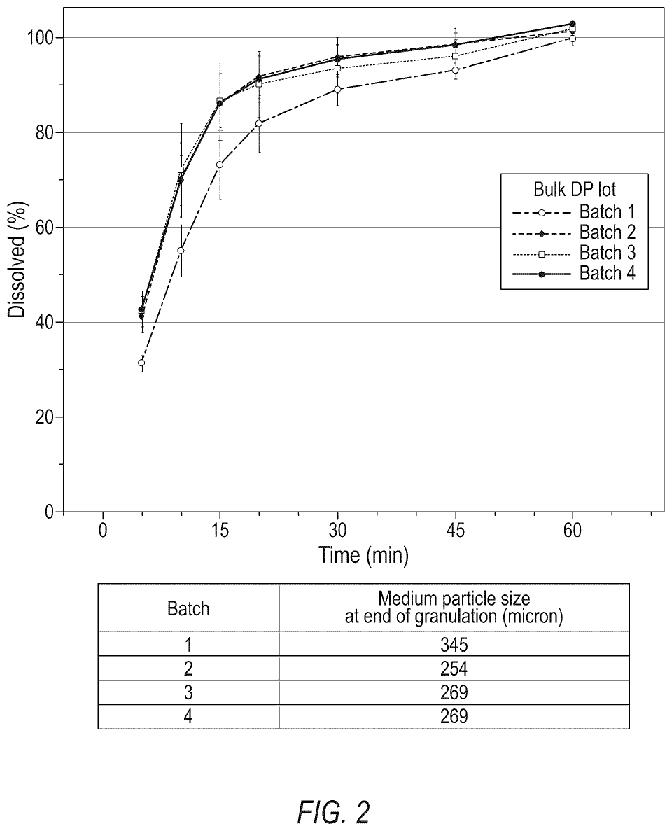

The present disclosure encompasses the recognition that intragranular particle size plays an important role in the dissolution profile of the provided pharmaceutical compositions and therefore plays a role in the bioavailability of maribavir. For example, dissolution studies on maribavir granules, e.g., coated intragranular compositions, at pH 2.5 (50 mM phosphate buffer) show that median particle sizes of 254 and 269 microns have a % dissolution 10-15% higher than median particles sizes of 345 microns after 5, 10, 15, and 20 minutes (see FIG. 2).

In some embodiments, the median particle diameter of an intragranular pharmaceutical composition (e.g., comprising maribavir, MCC, and crospovidone) is between about 100 and 400 microns. In some embodiments, the median particle diameter of an intragranular pharmaceutical composition is between about 100 and 350 microns. In some embodiments, the median particle diameter of an intragranular pharmaceutical composition is between about 100 and 320 microns. In some embodiments, the median particle diameter of an intragranular pharmaceutical composition is between about 100 and 300 microns. In some embodiments, the median particle diameter of an intragranular pharmaceutical composition is between about 100 and 280 microns. In some embodiments, the median particle diameter of an intragranular pharmaceutical composition is between about 100 and 260 microns. In some embodiments, the median particle diameter of an intragranular pharmaceutical composition is between about 100 and 240 microns. In some embodiments, the median particle diameter of an intragranular pharmaceutical composition is between about 100 and 220 microns. In some embodiments, the median particle diameter of an intragranular pharmaceutical composition is between about 100 and 200 microns. In some embodiments, the median particle diameter of an intragranular pharmaceutical composition is between about 100 and 180 microns. In some embodiments, the median particle diameter of an intragranular pharmaceutical composition is between about 100 and 150 microns. In some embodiments, the median particle diameter of an intragranular pharmaceutical composition is between about 100 and 130 microns. In some embodiments, the median particle diameter of an intragranular pharmaceutical composition is about 150 microns or less.

As described above and herein, in some embodiments an intragranular composition is coated with an enteric coating. In some embodiments, the median particle diameter of a coated intragranular pharmaceutical composition is about 500 microns or less. In some embodiments, the median particle diameter of a coated intragranular pharmaceutical composition is about 400 microns or less. In some embodiments, the median particle diameter of a coated intragranular pharmaceutical composition is about 300 microns or less. In some embodiments, the median particle diameter of a coated intragranular pharmaceutical composition is about 225-350 microns. In some embodiments, the median particle diameter of a coated intragranular pharmaceutical composition is about 250-325 microns, about 250-310 microns, about 250-300 microns, about 250-290 microns, about 250-280 microns, about 250-270 microns, or about 225-300 microns. In some embodiments, the median particle diameter of a coated intragranular pharmaceutical composition is about 250-300 microns.

In some embodiments, the dv50 (50th percentile; median) particle size of a coated intragranular pharmaceutical composition comprises particles of about 250-400 microns. In some embodiments, the dv50 particle size of a coated intragranular pharmaceutical composition comprises particles of about 250-380 microns. In some embodiments, the dv50 particle size of a coated intragranular pharmaceutical composition comprises particles of about 250-350 microns. In some embodiments, the dv50 particle size of a coated intragranular pharmaceutical composition comprises particles of about 250-340 microns. In some embodiments, the dv50 particle size of a coated intragranular pharmaceutical composition comprises particles of about 250-320 microns. In some embodiments, the dv50 particle size of a coated intragranular pharmaceutical composition comprises particles of about 250-300 microns. In some embodiments, the dv50 particle size of a coated intragranular pharmaceutical composition comprises particles of about 260-400 microns. In some embodiments, the dv50 particle size of a coated intragranular pharmaceutical composition comprises particles of about 270-400 microns. In some embodiments, the dv50 particle size of a coated intragranular pharmaceutical composition comprises particles of about 280-400 microns. In some embodiments, the dv50 particle size of a coated intragranular pharmaceutical composition comprises particles of about 290-400 microns. In some embodiments, the dv50 particle size of a coated intragranular pharmaceutical composition comprises particles of about 300-400 microns. In some embodiments, the dv50 particle size of a coated intragranular pharmaceutical composition comprises particles of about 320-400 microns. In some embodiments, the dv50 particle size of a coated intragranular pharmaceutical composition comprises particles of about 350-400 microns. In some embodiments, the dv50 particle size of a coated intragranular pharmaceutical composition comprises particles of about 150-260 microns. In some embodiments, the dv50 particle size of a coated intragranular pharmaceutical composition comprises particles of about 175-260 microns. In some embodiments, the dv50 particle size of a coated intragranular pharmaceutical composition comprises particles of about 200-260 microns. In some embodiments, the dv50 particle size of a coated intragranular pharmaceutical composition comprises particles of about 225-260 microns. In some embodiments, the dv50 particle size of a coated intragranular pharmaceutical composition comprises particles of about 250-260 microns. In some embodiments, the dv50 particle size of a coated intragranular pharmaceutical composition comprises particles of about 250, 251, 252, 253, 254, 255, 256, 257, 257, 258, 259, 260, 261, 262, 263, 264, 265, 266, 267, 268, 269, or 270 microns. In some embodiments, the dv50 particle size of a coated intragranular pharmaceutical composition comprises particles of about 250 microns. In some embodiments, the dv50 particle size of a coated intragranular pharmaceutical composition comprises particles of about 251 microns. In some embodiments, the dv50 particle size of a coated intragranular pharmaceutical composition comprises particles of about 252 microns. In some embodiments, the dv50 particle size of a coated intragranular pharmaceutical composition comprises particles of about 253 microns. In some embodiments, the dv50 particle size of a coated intragranular pharmaceutical composition comprises particles of about 254 microns. In some embodiments, the dv50 particle size of a coated intragranular pharmaceutical composition comprises particles of about 255 microns. In some embodiments, the dv50 particle size of a coated intragranular pharmaceutical composition comprises particles of about 256 microns. In some embodiments, the dv50 particle size of a coated intragranular pharmaceutical composition comprises particles of about 257 microns. In some embodiments, the dv50 particle size of a coated intragranular pharmaceutical composition comprises particles of about 258 microns. In some embodiments, the dv50 particle size of a coated intragranular pharmaceutical composition comprises particles of about 259 microns. In some embodiments, the dv50 particle size of a coated intragranular pharmaceutical composition comprises particles of about 260 microns. In some embodiments, the dv50 particle size of a coated intragranular pharmaceutical composition comprises particles of about 261 microns. In some embodiments, the dv50 particle size of a coated intragranular pharmaceutical composition comprises particles of about 262 microns. In some embodiments, the dv50 particle size of a coated intragranular pharmaceutical composition comprises particles of about 263 microns. In some embodiments, the dv50 particle size of a coated intragranular pharmaceutical composition comprises particles of about 264 microns. In some embodiments, the dv50 particle size of a coated intragranular pharmaceutical composition comprises particles of about 265 microns. In some embodiments, the dv50 particle size of a coated intragranular pharmaceutical composition comprises particles of about 266 microns. In some embodiments, the dv50 particle size of a coated intragranular pharmaceutical composition comprises particles of about 267 microns. In some embodiments, the dv50 particle size of a coated intragranular pharmaceutical composition comprises particles of about 268 microns. In some embodiments, the dv50 particle size of a coated intragranular pharmaceutical composition comprises particles of about 269 microns. In some embodiments, dv50 particle size of a coated intragranular pharmaceutical composition comprises particles of about 270 microns.

In some embodiments, the dv90 (90th percentile) particle size of a coated intragranular pharmaceutical composition comprises particles of about 350-500 microns. In some embodiments, the dv90 particle size of a coated intragranular pharmaceutical composition comprises particles of about 350-475 microns. In some embodiments, the dv90 particle size of a coated intragranular pharmaceutical composition comprises particles of about 350-450 microns. In some embodiments, the dv90 particle size of a coated intragranular pharmaceutical composition comprises particles of about 350-425 microns. In some embodiments, the dv90 particle size of a coated intragranular pharmaceutical composition comprises particles of about 350-400 microns. In some embodiments, the dv90 particle size of a coated intragranular pharmaceutical composition comprises particles of about 350-375 microns.

In some embodiments, the dv10 (10th percentile) particle size of a coated intragranular pharmaceutical composition comprises particles of about 100-300 microns. In some embodiments, the dv10 particle size of a coated intragranular pharmaceutical composition comprises particles of about 120-300 microns. In some embodiments, the dv10 particle size of a coated intragranular pharmaceutical composition comprises particles of about 150-300 microns. In some embodiments, the dv10 particle size of a coated intragranular pharmaceutical composition comprises particles of about 175-300 microns. In some embodiments, the dv10 particle size of a coated intragranular pharmaceutical composition comprises particles of about 200-300 microns. In some embodiments, the dv10 particle size of a coated intragranular pharmaceutical composition comprises particles of about 225-300 microns. In some embodiments, the dv10 particle size of a coated intragranular pharmaceutical composition comprises particles of about 250-300 microns. In some embodiments, the dv10 particle size of a coated intragranular pharmaceutical composition comprises particles of about 260-300 microns. In some embodiments, the dv10 particle size of a provided pharmaceutical composition comprises particles of about 270-300 microns. In some embodiments, the dv10 particle size of a coated intragranular pharmaceutical composition comprises particles of about 280-300 microns.

### Coated Intragranular Dispersion in Extragranular Matrix

In some embodiments, a provided intragranular composition (e.g., maribavir and excipients within an enteric layer) is further dispersed in extragranular components. In some embodiments, a provided pharmaceutical composition of intragranular components (e.g., maribavir, a diluent, and a disintegrant) and selectively soluble polymer coating is further dispersed in extragranular components forming a solid-sol. In some embodiments, a provided pharmaceutical composition is a coated intragranular composition further dispersed in extragranular components forming a solid-sol. In some embodiments, provided pharmaceutical composition is a solid-sol, for dispersion in water, comprising: an active pharmaceutical ingredient, a diluent, and a disintegrant within an enteric envelope, further comprising a diluent, a suspending agent, a sweetener, and a flavorant. In some embodiments, a provided pharmaceutical composition is a solid-sol, for dispersion in water, comprising: maribavir, microcrystalline cellulose, crospovidone, Eudragit® E PO, mannitol, carboxymethylcellulose sodium, sucralose, and a flavorant.

### Input Particle Size

### Maribavir Particle Size

In some embodiments, the median particle size (i.e., 50^{th} percentile or dv50) of maribavir is about 1-24 microns. In some embodiments, the median particle size of maribavir is about 1-18 microns. In some embodiments, the median particle size of maribavir is about 1-12 microns. In some embodiments, the median particle size of maribavir is about 1-6 microns. In some embodiments, the median particle size of maribavir is about 1-3 microns. In some embodiments, the median particle size of maribavir is about 3-18 microns. In some embodiments, the median particle size of maribavir is about 6-18 microns.

In some embodiments, the dv90 (i.e., 90^{th} percentile) particle size of maribavir is about 90 microns or less. In some embodiments, the dv90 particle size of maribavir is about 60 microns or less. In some embodiments, the dv90 particle size of maribavir is about 40 microns or less. In some embodiments, the dv90 particle size of maribavir is about 30 microns or less. In some embodiments, the dv90 particle size of maribavir is 25 microns or less. In some embodiments, the dv90 particle size of maribavir is 15 microns or less. In some embodiments, the dv90 particle size of maribavir is 10 microns or less.

### Microcrystalline Cellulose

In some embodiments, the dv10 (i.e., 10^{th} percentile) particle size of microcrystalline cellulose (MCC), for example, Avicel® PH-105, is about 1-50 microns. In some embodiments, the dv10 particle size of MCC is about 1-40 microns. In some embodiments, the dv10 particle size of MCC is about 1-30 microns. In some embodiments, the dv10 particle size of MCC is about 1-20 microns. In some embodiments, the dv10 particle size of MCC is about 1-10 microns. In some embodiments, the dv10 particle size of MCC is about 1-5 microns.

In some embodiments, the dv50 (i.e., 50^{th} percentile) particle size of MCC is about 2-90 microns. In some embodiments, the dv50 particle size of MCC is about 5-80 microns. In some embodiments, the dv50 particle size of MCC is about 5-60 microns. In some embodiments, the dv50 particle size of MCC is about 5-50 microns. In some embodiments, the dv50 particle size of MCC is about 5-40 microns. In some embodiments, the dv50 particle size of MCC is about 5-30 microns. In some embodiments, the dv50 particle size of MCC is about 5-20 microns. In some embodiments, the dv50 particle size of MCC is about 10-50 microns. In some embodiments, the dv50 particle size of MCC is about 15-40 microns. In some embodiments, the dv50 particle size of MCC is about 15-30 microns. In some embodiments, the dv50 particle size of MCC is about 10-30 microns. In some embodiments, the dv50 particle size of MCC is about 20-30 microns. In some embodiments, the dv50 particle size of MCC is about 25-30 microns.

In some embodiments, the dv90 (i.e., 90^{th} percentile) particle size of MCC is about 10-150 microns. In some embodiments, the dv90 (i.e., 90^{th} percentile) particle size of MCC is about 10-120 microns. In some embodiments, the dv90 (i.e., 90^{th} percentile) particle size of MCC is about 10-90 microns. In some embodiments, the dv90 (i.e., 90^{th} percentile) particle size of MCC is about 10-75 microns. In some embodiments, the dv90 (i.e., 90^{th} percentile) particle size of MCC is about 10-60 microns. In some embodiments, the dv90 (i.e., 90^{th} percentile) particle size of MCC is about 10-50 microns. In some embodiments, the dv90 (i.e., 90^{th} percentile) particle size of MCC is about 10-40 microns. In some embodiments, the dv90 (i.e., 90^{th} percentile) particle size of MCC is about 20-90 microns. In some embodiments, the dv90 (i.e., 90^{th} percentile) particle size of MCC is about 20-75 microns. In some embodiments, the dv90 (i.e., 90^{th} percentile) particle size of MCC is about 20-60 microns. In some embodiments, the dv90 (i.e., 90^{th} percentile) particle size of MCC is about 30-60 microns. In some embodiments, the dv90 (i.e., 90^{th} percentile) particle size of MCC is about 40-60 microns.

### Crospovidone

In some embodiments, the dv10 (i.e., 10^{th} percentile) particle size of crospovidone, for example, Polyplasdone™ XL-10, is about 2-48 microns. In some embodiments, the dv10 particle size of crospovidone is about 8-48 microns. In some embodiments, the dv10 particle size of crospovidone is about 16-48 microns. In some embodiments, the dv10 particle size of crospovidone is about 8-48 microns. In some embodiments, the dv10 particle size of crospovidone is about 2-36 microns. In some embodiments, the dv10 particle size of crospovidone is about 2-24 microns. In some embodiments, the dv10 particle size of crospovidone is about 2-12 microns. In some embodiments, the dv10 particle size of crospovidone is about 2-36 microns. In some embodiments, the dv10 particle size of crospovidone is about 2-16 microns. In some embodiments, the dv10 particle size of crospovidone is about 4-16 microns. In some embodiments, the dv10 particle size of crospovidone is about 8-16 microns. In some embodiments, the dv10 particle size of crospovidone is about 10-16 microns. In some embodiments, the dv10 particle size of crospovidone is about 6-12 microns. In some embodiments, the dv10 particle size of crospovidone is about 8-12 microns. In some embodiments, the dv10 particle size of crospovidone is about 10-12 microns.

In some embodiments, the dv50 (i.e., 50^{th} percentile) particle size of crospovidone is about 5-120 microns. In some embodiments, the dv50 particle size of crospovidone is about 10-120 microns. In some embodiments, the dv50 particle size of crospovidone is about 15-120 microns. In some embodiments, the dv50 particle size of crospovidone is about 20-120 microns. In some embodiments, the dv50 particle size of crospovidone is about 5-100 microns. In some embodiments, the dv50 particle size of crospovidone is about 5-80 microns. In some embodiments, the dv50 particle size of crospovidone is about 5-60 microns. In some embodiments, the dv50 particle size of crospovidone is about 5-40 microns. In some embodiments, the dv50 particle size of crospovidone is about 10-50 microns. In some embodiments, the dv50 particle size of crospovidone is about 10-40 microns. In some embodiments, the dv50 particle size of crospovidone is about 10-30 microns. In some embodiments, the dv50 particle size of crospovidone is about 20-30 microns. In some embodiments, the dv50 particle size of crospovidone is about 25-30 microns. In some embodiments, the dv50 particle size of crospovidone is about 20-25 microns.

In some embodiments, the dv90 (i.e., 90^{th} percentile) particle size of crospovidone is about 10-250 microns. In some embodiments, the dv90 particle size of crospovidone is about 30-250 microns. In some embodiments, the dv90 particle size of crospovidone is about 50-250 microns. In some embodiments, the dv90 particle size of crospovidone is about 80-250 microns. In some embodiments, the dv90 particle size of crospovidone is about 10-200 microns. In some embodiments, the dv90 particle size of crospovidone is about 10-150 microns. In some embodiments, the dv90 particle size of crospovidone is about 10-100 microns. In some embodiments, the dv90 particle size of crospovidone is about 10-50 microns. In some embodiments, the dv90 particle size of crospovidone is about 30-250 microns. In some embodiments, the dv90 particle size of crospovidone is about 50-200 microns. In some embodiments, the dv90 particle size of crospovidone is about 60-180 microns. In some embodiments, the dv90 particle size of crospovidone is about 70-150 microns. In some embodiments, the dv90 particle size of crospovidone is about 75-130 microns. In some embodiments, the dv90 particle size of crospovidone is about 80-120 microns. In some embodiments, the dv90 particle size of crospovidone is about 85-110 microns. In some embodiments, the dv90 particle size of crospovidone is about 85-100 microns. In some embodiments, the dv90 particle size of crospovidone is about 25-100 microns. In some embodiments, the dv90 particle size of crospovidone is about 30-95 microns. In some embodiments, the dv90 particle size of crospovidone is about 35-85 microns. In some embodiments, the dv90 particle size of crospovidone is about 40-80 microns. In some embodiments, the dv90 particle size of crospovidone is about 45-75 microns.

### Extragranular Components

In some embodiments, a provided pharmaceutical composition comprises a coated intragranular composition within an extragranular matrix (e.g., composition). In some embodiments, the extragranular composition comprises a diluent, a suspending agent, a sweetener, and a flavor. In some embodiments, the extragranular composition comprises mannitol (e.g., Pearlitol® 100SD, microcrystalline cellulose and carboxymethylcellulose sodium (e.g., Avicel® CL-611), sucralose (Emprove®), and a flavorant (e.g., fruit flavor).

In some embodiments, the extragranular matrix comprises excipients that inhibit particle segregation (e.g., a sugar alcohol such as mannitol). In some embodiments, the extragranular matrix comprises flavorants that provide flavor masking during liquid-suspension oral administration. In some embodiments, the extragranular matrix comprises a sweetener or sweeteners. In some embodiments, the extragranular matrix comprises a suspending agent. In some embodiments, the extragranular matrix comprises a sugar alcohol. In some embodiments, the extragranular matrix comprises mannitol.

### Particle Segregation

As discussed above, in some embodiments a provided pharmaceutical composition comprises active pharmaceutical ingredients and pharmaceutically acceptable excipients. It will be appreciated that a coated intragranular pharmaceutical composition will have a statistic distribution of particle sizes, shapes, densities, etc. Upon continued exposure to vibration, movement, humidity, gravity, and relative airflow, particles of similar sizes tend to group together by the phenomenon of particle segregation. Particle segregation can lead to local statistical distributions of particle sizes, densities, shapes, or some other characteristic and thus different active pharmaceutical quantities within each subpopulation (e.g., dose) of the pharmaceutical composition (e.g., coated intragranular pharmaceutical composition), become separated in space instead of remaining a single homogenized statistical distribution. Among other things, to maintain uniformity of particulate pharmaceutical compositions throughout manufacturing and dosing, such particle segregation behaviors can be controlled. The present disclosure encompasses the recognition that the addition of mannitol (e.g., as a diluent) is particularly useful to controlling particle segregation of provided pharmaceutical compositions. Methods of measuring particle segregation are known to the skilled person, and include by way of non-limiting example ASTM standards D6940 and D6941.

### Flowability

In some embodiments, flowability is the relative movement of bulk particles (e.g., a pharmaceutical composition) among neighboring particles or along a container wall or a surface. In some embodiments, various characterization dimensions can express flowability. In some embodiments, flowability is measured by angle of repose, compressibility index (Hausner ratio), flow in a rotating drum and through an orifice, shear cell, or rheometry. In some embodiments, flowability is expressed by way of a flowing angle or dynamic cohesive index, among others. The present disclosure encompasses the recognition that the addition of mannitol (e.g., as a diluent) is particularly useful to improving flowability of provided pharmaceutical compositions. In some embodiments, the flowability may be improved by use of a filling device with vibration functionality.

### Mannitol

In some embodiments, an extragranular matrix (e.g., composition) of a provided pharmaceutical composition comprises a particle segregation inhibitor. In some embodiments, a particle segregation inhibitor is mannitol.

In some embodiments, an extragranular matrix of a provided pharmaceutical composition comprises a diluent particle segregation inhibitor. In some embodiments, an extragranular matrix of a provided pharmaceutical composition comprises a sugar alcohol. In some embodiments, an extragranular matrix of a provided pharmaceutical composition comprises mannitol. In some embodiments, an extragranular matrix of a provided pharmaceutical composition comprises spray-dried mannitol.

In some embodiments, the dv10 (i.e., 10^{th} percentile) particle size of mannitol, for example, Pearlitol® 100SD, is about 70 microns or less. In some embodiments, the dv10 particle size of mannitol is about 60 microns or less. In some embodiments, the dv10 particle size of mannitol is about 50 microns or less. In some embodiments, the dv10 particle size of mannitol is about 40 microns or less. In some embodiments, the dv10 particle size of mannitol is about 30 microns or less. In some embodiments, the dv10 particle size of mannitol is about 20 microns or less. In some embodiments, the dv10 particle size of mannitol is about 15 microns or less. In some embodiments, the dv10 particle size of mannitol is about 10 microns or less.

In some embodiments, the dv50 (i.e., 50^{th} percentile) particle size of mannitol is between about 30 and 180 microns. In some embodiments, the dv50 particle size of mannitol is between about 50 and 150 microns. In some embodiments, the dv50 particle size of mannitol is between about 70 and 130 microns. In some embodiments, the dv50 particle size of mannitol is between about 80 and 120 microns. In some embodiments, the dv50 particle size of mannitol is between about 90 and 110 microns. In some embodiments, the dv50 particle size of mannitol is between about 95 and 95 microns. In some embodiments, the dv50 particle size of mannitol is between about 50 and 110 microns. In some embodiments, the dv50 particle size of mannitol is between about 60 and 110 microns. In some embodiments, the dv50 particle size of mannitol is between about 70 and 110 microns. In some embodiments, the dv50 particle size of mannitol is between about 80 and 110 microns. In some embodiments, the dv50 particle size of mannitol is between about 90 and 150 microns. In some embodiments, the dv50 particle size of mannitol is between about 90 and 140 microns. In some embodiments, the dv50 particle size of mannitol is between about 90 and 130 microns. In some embodiments, the dv50 particle size of mannitol is between about 90 and 120 microns. In some embodiments, the dv50 particle size of mannitol is about 100 microns.

In some embodiments, the dv90 (90^{th} percentile) particle size of mannitol is about 150 microns or more. In some embodiments, the dv90 particle size of mannitol is about 160 microns or more. In some embodiments, the dv90 particle size of mannitol is about 170 microns or more. In some embodiments, the dv90 particle size of mannitol is about 180 microns or more. In some embodiments, the dv90 particle size of mannitol is about 185 microns or more. In some embodiments, the dv90 particle size of mannitol is about 195 microns or more. In some embodiments, the dv90 particle size of mannitol is about 200 microns. In some embodiments, the dv90 particle size of mannitol is about 210 microns. In some embodiments, the dv90 particle size of mannitol is about 225 microns.

In some embodiments, a provided pharmaceutical composition comprises spray-dried mannitol. In some embodiments, a provided pharmaceutical composition comprises amorphous mannitol.

In some embodiments, provided pharmaceutical compositions are composed of maribavir and excipients in some ratios are expressed in w/w %. In some embodiments, provided pharmaceutical compositions are composed of about 25-40% maribavir, about 10-22% microcrystalline cellulose, about 0.5-4% crospovidone, with the remaining amount comprising a coating polymer (e.g., an amino methacrylate copolymer). In some embodiments, provided pharmaceutical compositions are composed of about 25-40% maribavir, about 10-22% microcrystalline cellulose, about 0.5-4% crospovidone, and about 34-65% coating polymer (e.g., Eudragit® E PO). In some embodiments, provided pharmaceutical compositions are composed of about 30-40% maribavir, about 15-22% microcrystalline cellulose, about 1-4% crospovidone, and about 34-54% coating polymer (e.g., Eudragit® E PO). In some embodiments, provided pharmaceutical compositions are composed of about 25-37% maribavir, about 10-18% microcrystalline cellulose, about 0.5-2.5% crospovidone, and about 42-65% coating polymer (e.g., Eudragit® E PO). In some embodiments, provided pharmaceutical compositions are composed of about 31-37% maribavir, about 14-18% microcrystalline cellulose, about 1.5-2.5% crospovidone, and about 42-54% coating polymer (e.g., Eudragit® E PO). In some embodiments, provided pharmaceutical compositions are composed of about 32.5% maribavir, about 15.5% microcrystalline cellulose, about 2% crospovidone, and about 50% coating polymer (e.g., Eudragit® E PO). In some embodiments, provided pharmaceutical compositions are composed of about 36.1% maribavir, about 17.2% microcrystalline cellulose, about 2.2% crospovidone, and about 44.4% coating polymer (e.g., Eudragit® E PO). In some embodiments, provided pharmaceutical compositions are composed of about 10-25% maribavir, about 5-40% suspending agent (e.g., microcrystalline cellulose and carboxymethylcellulose sodium), about 0.5-2% crospovidone, about 15-25% coating polymer (e.g., Eudragit® E PO), about 5-15% mannitol, about 5-25% sweetener (e.g., sucralose), and about 2-10% flavorant (e.g., fruit flavor). In some embodiments, provided pharmaceutical compositions are composed of about 10-25% maribavir, about 5-40% suspending agent (e.g., microcrystalline cellulose and carboxymethylcellulose sodium), about 0.5-2% crospovidone, about 15-25% coating polymer (e.g., Eudragit® E PO), about 5-15% mannitol, about 5-25% sweetener (e.g., sucralose), and about 2-10% flavorant (e.g., fruit flavor). In some embodiments, provided pharmaceutical compositions are composed of about 12-22% maribavir, about 20-40% suspending agent (e.g., microcrystalline cellulose and carboxymethylcellulose sodium), about 0.5-1.5% crospovidone, about 18-23% coating polymer (e.g., Eudragit® E PO), about 8-12% mannitol, about 10-20% sweetener (e.g., sucralose), and about 4-8% flavorant (e.g., fruit flavor). In some embodiments, provided pharmaceutical compositions are composed of about 15-20% maribavir, about 25-40% suspending agent (e.g., microcrystalline cellulose and carboxymethylcellulose sodium), about 1% crospovidone, about 19-21% coating polymer (e.g., Eudragit® E PO), about 9-11% mannitol, about 11-15% sweetener (e.g., sucralose), and about 5-7% flavorant (e.g., fruit flavor). In some embodiments, provided pharmaceutical compositions are composed of about 15-20% maribavir, about 30-35% suspending agent (e.g., microcrystalline cellulose and carboxymethylcellulose sodium), about 1% crospovidone, about 19-21% coating polymer (e.g., Eudragit® E PO), about 9-11% mannitol, about 11-15% sweetener (e.g., sucralose), and about 5-7% flavorant (e.g., fruit flavor). In some embodiments, provided pharmaceutical compositions are composed of about 16.6% maribavir, about 33% suspending agent (e.g., microcrystalline cellulose and carboxymethylcellulose sodium), about 1% crospovidone, about 20.3% coating polymer (e.g., Eudragit® E PO), about 10% mannitol, about 13.3% sweetener (e.g., sucralose), and about 5.8% flavorant (e.g., fruit flavor).

In some embodiments, a provided pharmaceutical composition is composed of about 50 mg maribavir, about 23.9 mg microcrystalline cellulose (Avicel® PH105), about 3.1 mg crospovidone (Polyplasdone™ XL-10), about 61.5 mg Eudragit® E PO ReadyMix, about 30.1 mg mannitol, about 75 mg microcrystalline cellulose and carboxymethylcellulose sodium (Avicel® CL-611), about 40 mg sucralose (Emprove), and about 17.5 mg of flavorant (e.g., fruit flavor).

In some embodiments, a provided pharmaceutical composition is composed of about 200 mg maribavir, about 95.6 mg microcrystalline cellulose (Avicel® PH105), about 12.4 mg crospovidone (Polyplasdone™ XL-10), about 246 mg Eudragit® E PO ReadyMix, about 120.4 mg mannitol, about 300 mg microcrystalline cellulose and carboxymethylcellulose sodium (Avicel® CL-611), about 160 mg sucralose (Emprove), and about 70 mg of flavorant (e.g., fruit flavor).

Also provided are solid form preparations that are intended to be converted, shortly before use, to liquid form preparations for oral administration. Such liquid forms include solutions, suspensions, dispersions, colloids, sols, and emulsions. These preparations may contain, in addition to the active component, colorants, flavorants, stabilizers, buffers, artificial and natural sweeteners, dispersants, thickeners, solubilizing agents, and the like.

In some embodiments, a provided maribavir formulation is an oral liquid formulation. In some embodiments, such formulations are prepared by suspending a provided solid pharmaceutical composition in a liquid (e.g., water).

A provided pharmaceutical preparation can be in unit dosage form. In such form the preparation can be subdivided into unit doses containing appropriate quantities of the active component. The unit dosage form can be a packaged preparation, the package containing discrete quantities of preparation.

Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, microcrystalline cellulose, carboxymethylcellulose, hydroxy propyl cellulose, chondroitin sulfate and salts thereof, hyaluronic acid and salts thereof, and combinations of the foregoing.

In some embodiments, a provided pharmaceutical composition further comprises a viscosity enhancer, viscosity modifier, or suspending agent (e.g., microcrystalline cellulose and carboxymethylcellulose sodium, methylcellulose, hydroxyethyl cellulose, hypromellose, or a combination thereof). In some embodiments, a suspending agent is a mixture of microcrystalline cellulose and carboxymethylcellulose sodium (e.g., Avicel® CL-611).

In some embodiments, a provided pharmaceutical composition for liquid oral administration is packaged within a stickpack. In some embodiments, a stickpack container is resistant to air and moisture. In some embodiments, a stickpack container comprises laminated paper or aluminum. In some embodiments, a stickpack container is stable at room temperature without degradation of the provided pharmaceutical composition for liquid oral administration. Stickpacks are a packaging solution for powder, granule, and other formulations, providing a convenient portable unit-dose solution for pharmaceuticals. Typically, a stickpack is tubular in shape with an elongated configuration, and often has continuous side seams.

### Methods of Manufacture

The present disclosure further provides methods for manufacturing ready-to-use pharmaceutical composition for liquid oral administration, comprising following steps:
i) granulating an active pharmaceutical ingredient and other excipients to form an intragranular composition;
ii) coating the intragranular composition with a selectively soluble polymer to form a coated intragranular composition;
iii) blending the coated intragranular composition, diluent, sweetener, flavorant, and other excipients to form a blended composition;
iv) filling and packaging the blended composition;
   wherein, median particle diameter of the intragranular composition is between about 100 and 400 microns, wherein median particle diameter of the coated intragranular composition is less than 500 microns, and wherein dv50 (i.e., 50^{th} percentile) particle size of the diluent is between about 30 and 180 microns.

In some embodiments, the provided methods are methods for manufacturing ready-to-use pharmaceutical composition for liquid oral administration, comprising following steps:
i) granulating an active pharmaceutical ingredient and other excipients to form an intragranular composition;
ii) coating the intragranular composition with a selectively soluble polymer to form a coated intragranular composition;
iii) blending the coated intragranular composition, mannitol, sweetener, flavorant, and other excipients to form a blended composition;
iv) filling and packaging the blended composition;
   wherein, median particle diameter of the intragranular composition is between about 100 and 400 microns, and wherein median particle diameter of the coated intragranular composition is less than 500 microns, wherein dv50 (i.e., 50^{th} percentile) particle size of mannitol is between about 30 and 180 microns.

In some embodiments, the provided methods are methods for manufacturing ready-to-use pharmaceutical composition for liquid oral administration, comprising following steps:
i) granulating maribavir and other excipients to form an intragranular composition;
ii) coating the intragranular composition with a selectively soluble polymer to form a coated intragranular composition;
iii) blending the coated intragranular composition, mannitol, sweetener, flavorant, and other excipients to form a blended composition;
iv) filling and packaging the blended composition;
   wherein, median particle diameter of the intragranular composition is between about 100 and 400 microns, wherein median particle diameter of the coated intragranular composition is less than 500 microns, and wherein dv50 (i.e., 50^{th} percentile) particle size of mannitol is between about 30 and 180 microns.

In some embodiments, the provided methods of manufacturing further comprising a step of suspending the pharmaceutical composition in a liquid vehicle (e.g., water) prior to oral administration.

In some embodiments, the provided methods of manufacturing further comprise filling the blended composition into a stickpack. In some embodiments, the provided methods of manufacturing further comprise packaging the blended composition in a stickpack. The present compositions and methods have been shown to provide consistency in fill weight and a consistent dosage in each stickpack.

In some embodiments, the provided methods of manufacturing form a coated intragranular composition as described above and herein (e.g., where the median particle diameter of the coated intragranular composition is about 225-350 microns, about 250-325 microns, about 250-300 microns, about 250-290 microns, about 250-280 microns, about 250-270 microns, or about 225-300 microns).

In some embodiments, the provided methods of manufacturing comprising coating the intragranular composition with a selectively soluble polymer, wherein the selectively soluble polymer is insoluble at neutral pH but soluble at acidic pH. In some embodiments, the selectively soluble polymer is Eudragit® E PO.

In some embodiments, the provided methods of manufacturing comprise a step of granulating maribavir and other excipients to form an intragranular composition. In some embodiments, granulating comprises wet granulation. In some embodiments, granulating comprises dry granulation.

In some embodiments, maribavir is micronized maribavir (e.g., with particle sizes described above and herein). In some embodiments, other excipients comprise a microcrystalline cellulose, e.g., as described above and herein. In some embodiments, other excipients comprise Avicel PH-105®. In some embodiments, other excipients comprise crospovidone, e.g., as described above and herein. In some embodiments, other excipients comprise Polyplasdone™ XL-10 Crospovidone.

In some embodiments, a step of granulating maribavir and other excipients to form an intragranular composition comprises: a) loading maribavir (e.g., micronized maribavir) and other excipients (e.g., a microcrystalline cellulose such as Avicel PH-105® and a crospovidone such as Polyplasdone™ XL-10 Crospovidone) onto a granulator; b) blending the loaded maribavir and other excipients; and c) spray drying the blended Maribavir and other excipients. In some embodiments, a step of granulating maribavir and other excipients to form an intragranular composition is performed as described in Example 4 for unsieved maribavir pellets. In some embodiments, the intragranular composition is an unsieved intragranular composition (e.g., an unsieved maribavir intragranular composition).

In some embodiments, e.g., prior to a step of coating the intragranular composition with a selectively soluble polymer to form a coated intragranular composition, the provided methods further comprise a step of filtering an unsieved intragranular composition (e.g., unsieved intragranular maribavir composition) to form a sieved intragranular composition (e.g., a sieved intragranular maribavir composition). In some embodiments, filtering is performed as described in Example 4 for sieved maribavir pellets.

In some embodiments, a step of coating the intragranular composition with a selectively soluble polymer to form a coated intragranular composition comprises: a) loading the intragranular composition (e.g., a sieved intragranular maribavir composition) and the selectively soluble polymer (e.g., Eudragit® E PO (ReadyMix)) onto a granulator; b) blending the loaded intragranular composition (e.g., a sieved maribavir intragranular composition) and the selectively soluble polymer (e.g., Eudragit® E PO (ReadyMix)); and c) spray drying the blended intragranular composition (e.g., a sieved maribavir intragranular composition) and the selectively soluble polymer (e.g., Eudragit® E PO (ReadyMix)). In some embodiments, a step of coating the intragranular composition with a selectively soluble polymer to form a coated intragranular composition is performed as described in Example 4 for coated maribavir pellets.

The following enumerated embodiments include aspects of the present disclosure:

### Enumerated Embodiments

1. A method of treating cytomegalovirus (CMV) infection or disease in a patient suffering therefrom comprising administering maribavir to the patient, wherein the patient is less than 18 years of age.
2. The method of embodiment 1, wherein the patient is at least 12 years of age and less than 18 years of age.
3. The method of embodiment 1, wherein the patient is less than 12 years of age.
4. The method of embodiment 1, wherein the patient is at least 6 years of age and less than 12 years of age.
5. The method of embodiment 1, wherein the patient is less than 6 years of age.
6. The method of any one of the preceding embodiments, wherein the patient's body weight is less than 35 kg.
7. The method of any of the preceding embodiments, wherein the patient's body weight is at least 25 kg.
8. The method of any one of the preceding embodiments, wherein the patient's body weight is less than 25 kg.
9. The method of any one of the preceding embodiments, wherein the patient's body weight is 10-25 kg.
10. The method of any one of the preceding embodiments, wherein the patient's body weight is at least 5 kg.
11. The method of any one of the preceding embodiments, comprising administering about 400 mg of maribavir orally to the patient twice daily.
12. The method of any one of the preceding embodiments, comprising administering about 200 mg of maribavir orally to the patient twice daily.
13. A method of treating cytomegalovirus (CMV) infection or disease in a patient suffering therefrom comprising orally administering maribavir to the patient, wherein the patient is at least 12 years of age and less than 18 years of age, and wherein the amount of maribavir administered is
   i) 100 mg twice daily for patients having a body weight of at least 10 kg and less than 14 kg,
   ii) 200 mg twice daily for patients having a body weight of at least 14 kg and less than 25 kg, or
   iii) 400 mg twice daily for patients having a body weight of at least 25 kg.
14. A method of treating cytomegalovirus (CMV) infection or disease in a patient suffering therefrom comprising orally administering maribavir to the patient, wherein the patient is at least 6 years of age and less than 12 years of age, and wherein the amount of maribavir administered is
   i) 100 mg twice daily for patients having a body weight of at least 10 kg and less than 14 kg,
   ii) 200 mg twice daily for patients having a body weight of at least 14 kg and less than 25 kg, or
   iii) 400 mg twice daily for patients having a body weight of at least 25 kg.
15. A method of treating cytomegalovirus (CMV) infection or disease in a patient suffering therefrom comprising orally administering maribavir to the patient, wherein the patient is less than 6 years of age, and wherein the amount of maribavir administered is
   i) 50 mg once daily for patients having a body weight of at least 5 kg and less than 7 kg,
   ii) 50 mg twice daily for patients having a body weight of at least 7 kg and less than 10 kg,
   iii) 100 mg twice daily for patients having a body weight of at least 10 kg and less than 14 kg,
   iv) 200 mg twice daily for patients having a body weight of at least 14 kg and less than 25 kg, or
   v) 400 mg twice daily for patients having a body weight of at least 25 kg.
16. The method of any one of the preceding embodiments, wherein maribavir is administered each day.
17. The method of any one of the preceding embodiments, wherein maribavir is administered daily for at least 2 weeks, at least 3 weeks, at least 4 weeks, at least 5 weeks, at least 6 weeks, at least 7 weeks, or at least 8 weeks.
18. The method of any one of the preceding embodiments, wherein maribavir is administered in a therapeutically effective amount.
19. The method of any one of the preceding embodiments, wherein the patient has a CMV deoxyribonucleic acid (DNA) screening value of ≥1365 International Units per milliliter (IU/mL) in whole blood or ≥455 IU/mL in plasma in 2 consecutive assessments within 14 days of the first dose maribavir, separated by at least 1 day, by quantitative polymerase chain reaction (qPCR).
20. The method of any one of the preceding embodiments, wherein the patient does not have CMV tissue invasive disease involving the central nervous system (CNS) or retina prior to treatment with maribavir.
21. The method of any one of the preceding embodiments, further comprising the step of confirming CMV viremia clearance in the patient.
22. The method of embodiment 21, wherein CMV viremia clearance is defined as having a plasma CMV DNA concentration below a lower limit of quantification (e.g., <137 International units per milliliter [IU/mL] in 2 consecutive postbaseline samples, separated by at least 5 days).
23. The method of any one of the preceding embodiments, comprising administering maribavir to the patient with or without food.
24. The method of any one of the preceding embodiments, wherein the maribavir is administered as 200 mg tablets.
25. The method of any one of the preceding embodiments, wherein the patient is refractory to treatment with one or more other drugs to treat CMV infection or disease.
26. The method of any one of the preceding embodiments, wherein the patient is refractory to treatment with one or more of ganciclovir, valganciclovir, cidofovir, or foscarnet.
27. The method of any one of the preceding embodiments, wherein the patient is refractory to treatment with genotypic resistance.
28. The method of any one of the preceding embodiments, wherein the patient is refractory to treatment without genotypic resistance.
29. The method of any one of the preceding embodiments, wherein the patient is a transplant recipient.
30. The method of any one of the preceding embodiments, wherein the patient is a hematopoietic stem cell transplant recipient.
31. The method of any one of the preceding embodiments, wherein the patient is a solid organ transplant recipient.
32. The method of any one of the preceding embodiments, wherein the patient is receiving or has received a cytochrome P450 3A4 (CYP3A4) inducer prior to administration of maribavir, and comprising the step of discontinuing administration of the CYP3 A4 inducer prior to administration of maribavir.
33. The method of embodiment 32, wherein the CYP3 A4 inducer is selected from the group consisting of rifampin, avasimibe, carbamazepine, phenytoin, rifabutin, phenobarbital, and St. John's wort.
34. The method of embodiment 32 or 33, wherein the CYP3A4 inducer is a strong CYP3A4 inducer.
35. The method of any one of embodiments 31-33, wherein the CYP3A4 inducer is selected from the group consisting of rifampin, rifabutin, and St. John's wort.
36. The method of any one of the preceding embodiments, wherein the patient is receiving or has received a cytochrome P450 3A4 (CYP3A4) inducer, and comprising the step of increasing the amount of maribavir administered to the patient.
37. The method of embodiment 36, wherein the CYP3 A4 inducer is selected from the group consisting of rifampin, avasimibe, carbamazepine, phenytoin, rifabutin, phenobarbital, and St. John's wort.
38. The method of embodiment 36 or 37, wherein the CYP3A4 inducer is selected from the group consisting of rifampin, carbamazepine, phenytoin, rifabutin, phenobarbital, and St. John's wort.
39. The method of any one of embodiments 36-38, wherein the CYP3A4 inducer is selected from the group consisting of carbamazepine, phenytoin, and phenobarbital.
40. The method of any one of the preceding embodiments, wherein the patient is receiving or has received an immunosuppressant.
41. The method of embodiment 40, further comprising the step of monitoring the levels of immunosuppressant after commencing administration of maribavir (e.g., with comparison to a reference or standard level).
42. The method of embodiment 40 or 41, further comprising the step of reducing the amount of the immunosuppressant administered to the patient.
43. The method of any one of embodiments 40-42, further comprising the step of monitoring the levels of immunosuppressant after discontinuing administration of maribavir (e.g., with comparison to a reference or standard level).
44 The method of embodiment 43, further comprising the step of increasing the amount of immunosuppressant administered to the patient (e.g., to the amount of immunosuppressant that was administered prior to commencing administration of maribavir).
45. The method of any one of embodiments 40-44, wherein immunosuppressant is selected from the group consisting of tacrolimus, cyclosporine, everolimus, sirolimus, prednisone, and mycophenolate.
46. The method of any one of embodiments 40-45, wherein immunosuppressant is selected from the group consisting of tacrolimus, cyclosporine, everolimus, and sirolimus.
47. The method of any one of the preceding embodiments, wherein the patient is receiving or has received digoxin.
48. The method of embodiment 47, further comprising the step of monitoring the levels of digoxin.
49. The method of embodiment 47 or 48, further comprising the step of reducing the amount of the digoxin administered to the patient.

### EXAMPLES

### General Preparation of Oral Liquid Formulations

Maribavir, microcrystalline cellulose, crospovidone are granulated with purified water in a fluidized rotor granulator to form spherical granules. The granules are dried in a fluid bed processor. The dried granules are sieved to collect granules of the desired size range. The granules are coated with an aqueous suspension of Eudragit® E PO ReadyMix and dried in a fluid bed processor. The coated granules are sieved to exclude agglomerates that may have formed. The polymer coated granules are combined with a sweetener, e.g., sucralose, a flavorant, e.g., fruit flavor, a diluent, e.g., a sugar alcohol, e.g., mannitol, and optionally a suspending agent (microcrystalline cellulose and carboxymethylcellulose sodium) to create a powder-for-oral suspension formulation (pharmaceutical composition for oral liquid administration). Powder-for-oral suspension formulations are suspended in water to be administered orally.

### Example 1: A Phase 3, Open-Label, Single-Arm, Repeated-Dose Study to Evaluate the Safety and Tolerability, Pharmacokinetics, and Antiviral Activity of Maribavir for the Treatment of Cytomegalovirus (CMV) Infection in Children and Adolescents Who have Received a Hematopoietic Stem Cell Transplant (HSCT) or a Solid Organ Transplant (SOT)

The main aim of this study is to find out the safety, tolerability and PK of maribavir for the treatment of CMV infection in children and teenagers after HSCT or SOT and to identify the optimal dose of maribavir using a 200 milligrams (mg) adult tablet formulation or other formulation, i.e., an oral suspension formulation, based on pharmacokinetics (PK) modeling. The participants will be treated with maribavir for 8 weeks. Participants need to visit their doctor during 12-week follow-up period.

### Arms and Interventions

| Arms | Intervention |
|---|---|
| Cohort 1: Maribavir 400 or 200 mg | Drug: Maribavir |
| Participants with greater than or equal to (>=) 12 to less than (<) | Participants will receive |
| 18 years of age will receive maribavir 400 milligrams (mg) (2*200 | maribavir. |
| mg tablets) twice daily (BID) based on body weight >=25 | For the 400 mg BID and |
| kilogram (kg), or 200 mg tablet BID based on body weight | 200 mg BID doses, |
| 14 to <25 kg, or 100 mg BID based on body weight 10 to <14 | participants will receive |
| kg orally for up to 8 weeks treatment period (Day 0/Week 0 to | tablet OR powder for oral |
| Day 56/Week 8). The dosing regimen will be based on the | suspension. For the 100 mg |
| participant's body weight and may be updated over the course | BID dose, the participants |
| of the study based on the internal interim analyses on PK, | will receive powder for oral |
| safety, and tolerability of at least 5 participants in each cohort. | suspension. |
| Cohort 2: Maribavir 400 or 200 mg | Drug: Maribavir |
| Participants with >=6 to <12 years of age will receive | Participants will receive |
| maribavir 400 mg (2*200 mg tablets) BID based on body | maribavir. |
| weight >=25 kg, or 200 mg tablet BID based on body weight | For the 400 mg BID and |
| 14 to <25 kg, or 100 mg BID based on body weight 10 to <14 | 200 mg BID doses, |
| kg orally for up to 8 weeks treatment period (Day 0/Week 0 to | participants will receive |
| Day 56/Week 8). The dosing regimen will be based on the | tablet OR powder for oral |
| participant's body weight and may be updated over the course | suspension. For the 100 mg |
| of the study based on the internal interim analyses on PK, | BID dose, the participants |
| safety, and tolerability of at least 5 participants in each cohort. | will receive powder for oral |
| | suspension. |
| Cohort 3: Maribavir | Drug: Maribavir |
| Participants with 0 to <6 years of age will receive | Participants will receive |
| maribavir 400 mg BID based on body weight >=25 kg, or 200 | Maribavir. |
| mg BID based on body weight 14 to <25 kg, or 100 mg BID based | For each of the Cohort 3 |
| on body weight 10 to <14 kg, or 50 mg BID based on body weight | doses, participants will |
| 7 to <10 kg, or 50 mg once daily (QD) based on body weight | receive the powder for oral |
| 5 to <7 kg. Individuals less than 5 kg will not be dosed. | suspension formulation. |

Powder for oral suspension will be provided in stickpacks containing 50 or 200 mg maribavir. The powder for oral suspension should be stored at ambient temperature up to 25° C. and should not be frozen or refrigerated.

### Methodology:

This is a Phase 3, multicenter, open-label, single-arm, repeated-dose study to evaluate the safety and tolerability, PK, acceptability and palatability, and antiviral activity of maribavir for the treatment of CMV infection in male and female children and adolescents (from 0 years to less than 18 years of age) after HSCT or SOT. This Phase 3 study aims to identify the optimal dose of maribavir using an oral suspension or 200 mg tablet formulation. An external data monitoring committee will be utilized to review accumulating safety and tolerability data generated in this study.

There will be 3 age cohorts including:
Cohort 1: subjects≥12 years to <18 years of age.
Cohort 2: subjects>6 years to <12 years of age.
Cohort 3: subjects from 0 years to <6 years of age

Cohorts 1 and 2 will enroll at least 10 HSCT subjects in each cohort, and Cohort 3 will enroll at least 13 HSCT subjects with 10 subjects 2 years to <6 years of age and 3 subjects 0 to <2 years of age.

Concurrent enrollment of the 3 age cohorts is planned; however, the initiation of Cohort 3 will begin when the powder for oral suspension becomes available. Within Cohorts 1, 2, and 3, staggered enrollment will be implemented to allow internal interim analysis of safety and PK data for at least 5 subjects who complete Week 1 (Day 7 [+2 days]) PK sample collection. The objective of this interim analysis is to confirm the dose selection, to allow for dose adjustment, and to monitor the safety and tolerability of maribavir within each cohort.

In each cohort, subjects will receive doses according to the dosing table and serial PK samples will be collected at Week 1 (Day 7 [+2 days]). The PK samples collected at Week 1 (Day 7 [+2 days]) in at least 5 subjects enrolled in each cohort will be analyzed with quick turnaround to compare the PK exposure obtained in each subject, as well as in the cohort as a whole, to the target PK ranges, to allow dose adjustment based on the observed PK, and to monitor safety and tolerability within each cohort. Once PK exposure is confirmed and safety data reviewed, enrollment of the rest of the cohort will proceed.

An additional interim analysis will be conducted after 33 asymptomatic HSCT subjects over the 3 cohorts complete the study (with at least 10 asymptomatic HSCT subjects each in Cohorts 1 and 2, and 13 asymptomatic HSCT subjects in Cohort 3 [including 3 asymptomatic HSCT subjects 0 years to <2 years of age]) to allow submission of data to regulatory agencies.

To be eligible for the study, subjects must have a documented CMV infection in whole blood or plasma, with a screening value of >1365 IU/mL in whole blood or >455 IU/mL in plasma in 2 consecutive assessments within 14 days of first dose of study drug (with the second assessment within 5 days prior to first dose), separated by at least 1 day, as determined by local laboratory quantitative polymerase chain reaction (qPCR) or comparable quantitative CMV DNA results. Results should be available before the subject receives the first dose of maribavir treatment to verify subject eligibility for the study. The same source (blood or plasma) and the same laboratory must be used for both assessments.

The study will have 3 periods: 1) up to a 2-week screening period, 2) an 8-week study treatment period, and 3) a 12 week follow-up period after the treatment period.

### Screening Period

Approximately 100 subjects will be screened. Eligibility will be determined during the screening period from Day-14 to Day 1.

### Treatment Period

During the 8-week treatment period (Day 1/Week 1 to Day 56/Week 8), maribavir will be administered BID following the age- and weight tier-based dosing scheme. Assessments to be performed at weekly study visits during treatment include: CMV DNA quantification testing; incidence of CMV disease; transplant graft function, graft versus host disease (GVHD), underlying disease, and opportunistic infection assessments; and concomitant medications and AE review. Pharmacokinetic, maribavir palatability evaluation, physical examination, vital sign assessment, electrocardiograms (ECGs), immunosuppressant drug level monitoring, clinical laboratory testing (hematology and chemistry), and urinalysis will be conducted at selected visits throughout the treatment period. Cytomegalovirus genotyping will be performed on subjects≥2 years of age at baseline (Visit 2/Day 1/Week 1), and 1 additional time point, if applicable. Some of the assessments will be limited in Cohort 3 due to blood draw volume limitations. For subjects in Cohort 3 who develop CMV CNS infection while on study treatment, an optional cerebrospinal fluid sampling will be considered for those undergoing a lumbar puncture as part of the diagnostic workup prior to discontinuation of the study treatment.

Taste/palatability assessment such as hedonic “faces” scales will also be completed by the subjects (if possible) or their parent(s) or legally authorized representative (LAR) at Weeks 1, 4, and 8.

If study treatment requires discontinuation prior to Week 8 per investigator's clinical judgment, alternative anti CMV treatment may be administered after study treatment discontinuation, if deemed necessary. For subjects who discontinue study treatment prior to Week 8 or withdraw consent during the treatment period, the end of treatment evaluations described for Week 8 in the schedule of assessments will be performed as completely as possible at the Study Visit on or immediately after the last dose of study drug. Subjects who discontinue treatment early but remain in the study will follow a modified schedule of assessments through the remaining weekly visits of the study treatment period and then enter the 12-week follow up period.

### Follow-Up Period

After completing the 8-week study treatment period, subjects will enter the 12-week follow-up period. During the follow-up period, study-specific evaluations will include central specialty laboratory CMV testing; incidence of CMV disease; transplant graft function, GVHD and underlying disease assessments; concomitant medications; and AE review for the first 4 weeks, then monthly for the next 2 visits.

Subjects who withdraw from the study during the follow-up period will perform the end-of-study evaluations and procedures for Week 20 (Follow-up Week 12) as soon as possible.

### Outcome Measures

### Primary Outcome Measure

1. Maximum Observed Plasma Concentration (Cmax) of Maribavir
   Cmax of maribavir will be evaluated.
   [Time Frame: Pre-dose, 0.5, 1.5, 3, 4, 6, and 8 hours post-dose on Day 7 (Week 1).]
2. Time to Maximum Observed Concentration (Tmax) of Maribavir
   Tmax of maribavir will be evaluated.
   [Time Frame: Pre-dose, 0.5, 1.5, 3, 4, 6, and 8 hours post-dose on Day 7 (Week 1).]
3. Minimum Plasma Concentration (Cmin) of Maribavir
   Cmin of maribavir will be evaluated.
   [Time Frame: Pre-dose, 0.5, 1.5, 3, 4, 6, and 8 hours post-dose on Day 7 (Week 1); Pre-dose on Day 28 (Week 4); Pre-dose and 2 to 4 hours post-dose on Day 56 (Week 8).]
4. Area Under the Plasma Concentration-Time Curve Over the 1 Dosing Interval of 12 Hours at
   Steady State (AUCO-tau) of Maribavir
   AUCO-tau of maribavir will be evaluated.
   [Time Frame: Pre-dose, 0.5, 1.5, 3, 4, 6, and 8 hours post-dose on Day 7 (Week 1).]
5. Half-Life (t½) of Maribavir t½ of maribavir will be evaluated.
   [Time Frame: Pre-dose, 0.5, 1.5, 3, 4, 6, and 8 hours post-dose on Day 7 (Week 1).]
6. Terminal Elimination Rate Constant (lambda-z) of Maribavir
   Lambda-z of maribavir will be evaluated.
   [Time Frame: Pre-dose, 0.5, 1.5, 3, 4, 6, and 8 hours post-dose on Day 7 (Week 1).]
7. Apparent Volume of Distribution (Vz/F) of Maribavir
   Vz/F of maribavir will be evaluated.
   [Time Frame: Pre-dose, 0.5, 1.5, 3, 4, 6, and 8 hours post-dose on Day 7 (Week 1).]
8. Apparent Oral Clearance (CL/F) of Maribavir
   CL/F of maribavir will be evaluated.
   [Time Frame: Pre-dose, 0.5, 1.5, 3, 4, 6, and 8 hours post-dose on Day 7 (Week 1).]
9. Number of Participants With Treatment-emergent Adverse Events (TEAEs) and Serious TEAEs
   An adverse event (AE) is any untoward medical occurrence in a clinical investigation participants administered a pharmaceutical product and that does not necessarily have a causal relationship with this treatment. A TEAE is defined as any event emerging or manifesting at or after the initiation of treatment with an investigational product or medicinal product or any existing event that worsens in either intensity or frequency following exposure to the investigational product or medicinal product. An SAE is any untoward medical occurrence (whether considered related to investigational product or not and at any dose) that at any dose results in death, life-threatening, requires inpatient hospitalization or prolongation of existing hospitalization, results in persistent or significant disability/incapacity, is a congenital abnormality or birth defect, an important medical event. TEAEs include serious and non-serious AEs.
   [Time Frame: From start of study drug administration up to follow-up (Week 20).]

### Secondary Outcome Measure

10. Percentage of Participants With Confirmed CMV viremia Clearance at Week 8
   Confirmed CMV viremia clearance is defined as plasma CMV DNA concentration below the lower limit of quantification (LLOQ) at a central specialty laboratory, in 2 consecutive postbaseline samples, separated by at least 5 days) at Week 8 regardless of the length of study treatment. Percentage of participants with confirmed CMV viremia clearance at Week 8 will be reported.
   [Time Frame: At Week 8.]
11. Percentage of Participants who Achieve Maintenance of Confirmed CMV Viremia Clearance and Symptom Control at Week 8 Through Weeks 12, 16 and 20
   Confirmed CMV viremia clearance is defined as plasma CMV DNA concentration below the lower LLOQ at a central specialty laboratory, in 2 consecutive postbaseline samples, separated by at least 5 days regardless of the length of study treatment. CMV infection symptom control is defined as resolution or improvement of tissue invasive disease or CMV syndrome for symptomatic participants at baseline, or no new symptoms for asymptomatic participants at baseline. Percentage of participants who achieve maintenance of confirmed CMV viremia clearance and symptom control at Week 8 through Weeks 12, 16 and 20 will be reported.
   [Time Frame: At Week 8 through Weeks 12, 16, and 20.]
12. Percentage of Participants With Confirmed Recurrence of CMV Viremia on Study Treatment and Off Study Treatment
   Confirmed recurrence of CMV viremia is defined as plasma CMV DNA concentration greater than or equal to (>=) LLOQ when assessed in 2 consecutive plasma samples at least 5 days apart, after achieving confirmed viremia clearance. Percentage of participants with confirmed recurrence of viremia on study treatment and off study treatment will be reported.
   [Time Frame: Up to Week 20.]
13. Time to First Confirmed Viremia Clearance
   Confirmed CMV viremia clearance is defined as plasma CMV DNA concentration below LLOQ when assessed at a central specialty laboratory, in 2 consecutive postbaseline samples, separated by at least 5 days. Time to first confirmed viremia clearance at any time during the study will be summarized using the Kaplan-Meier method.
   [Time Frame: Up to Week 20.]
14. Percentage of Participants With Confirmed Recurrence of CMV Viremia Treated With Administered Alternative Anti-CMV Treatment During 12-Week Follow-up Period
   Confirmed recurrence of CMV viremia is defined as plasma CMV DNA concentration>=LLOQ when assessed in 2 consecutive plasma samples at least 5 days apart, after achieving confirmed viremia clearance. Percentage of participants with confirmed recurrence of CMV viremia treated with alternative anti-CMV treatment in the 12-Week follow-up period in participants with confirmed viremia clearance will be reported.
   [Time Frame: At 12-Weeks of follow-up period (Week 9 to 20).]
15. Change From Baseline in Log 10 Plasma CMV Deoxyribonucleic Acid (DNA) Load
   Change from baseline in log 10 plasma CMV DNA on study after receiving study treatment by study week will be reported.
   [Time Frame: Baseline up to Week 20.]
16. Number of Participants who Develop CMV Resistance to Maribavir
   CMV DNA genotyping will be performed for the UL97, UL54, UL56, and UL27 genes known to confer resistance to conventional anti-CMV therapies or Maribavir. Number of participants who developed post-baseline CMV resistance to maribavir up to Week 12 will be reported.
   [Time Frame: Up to Week 12.]
17. Summary Scores for Palatability Assessment of Maribavir
   Palatability (taste, feel, smell, ease of swallowing and after-taste) is being measured using a 5-point facial hedonic scale correlated with a 100-point linear visual analogue scale (VAS) ranges from 0: very bad to 100: very good. Scores will be summarized descriptively at Weeks 1, 4 and 8.
   [Time Frame: At Weeks 1, 4, and 8.]

### Exploratory Endpoint:

Evaluate the graft outcomes (acute rejection, graft loss or failure, GVHD in HSCT subjects), and time to graft events per type of transplant.

### Eligibility

### Inclusion Criteria:

Parent/both parents or legally authorized representative (LAR) must provide signature of informed consent and there must be documentation of assent by the participant, as age appropriate, before completing any study-related procedures. During the COVID-19 public health emergency, informed consent from a potential or current trial participant may, if permitted by local laws and regulations, be obtained via electronic informed consent capabilities or an electronic face-to-face consent interview when these individuals are unable to travel to the site.
Be a male or female child or adolescent<18 years of age at the time of consent. For participants in Cohort 3 only, must have a gestational age of at least 38 weeks and a minimum weight of 5 kg.
Be a recipient of an SOT or an HSCT that is functioning at the time of screening.
Have a documented CMV infection, with a CMV deoxyribonucleic acid (DNA) screening value of >=1365 International Units per milliliter (IU/mL) in whole blood or >=455 IU/mL in plasma in 2 consecutive assessments separated by at least 1 day, as determined by local or central specialty laboratory quantitative polymerase chain reaction (qPCR) or comparable quantitative nucleic acid amplification test (qNAAT) results. Both samples must be taken within 14 days of first dose of study drug, with the second sample obtained within 5 days prior to first dose of study drug. The same laboratory and same sample type (whole blood or plasma) must be used for both assessments.
Have all the following results as part of screening laboratory assessments:
   Absolute neutrophil count>=500 per cubic millimeter (/mm\hat{ }3) (0.5×10\hat{ }9 per liter [/L])
   Platelet count>=15,000/mm\hat{ }3 (15×10\hat{ }9/L)
   Hemoglobin>=8 grams per deciliter (g/dL) (>=80 g/L)
Have an estimated glomerular filtration rate (creatinine-based Bedside Schwartz equation)>=30 milliliters per minute (mL/min)/1.73 meter square (m\hat{ }2).
Be a female of nonchildbearing potential. If a female of childbearing potential, have a negative serum human chorionic gonadotropin (hCG) or beta-human chorionic gonadotropin (β-hCG) pregnancy test at screening. Males, or nonpregnant, nonlactating females who are sexually active must agree to comply with the applicable contraceptive requirements of this protocol during the study treatment administration period and for 90 days after the last dose of study treatment.
Have life expectancy of >=8 weeks.
Be willing and have an understanding and ability to fully comply with the study procedures and restrictions defined in the protocol. For younger children, the parent/both parents or LAR must meet this criterion.

### Exclusion Criteria:

Have CMV tissue invasive disease involving the central nervous system (CNS) or retina as assessed by the investigator at the time of screening.
Have uncontrolled other type of infection as assessed by the investigator on the date of enrollment.
Have a history of clinically relevant alcohol or drug abuse that may interfere with treatment compliance or assessments with the protocol as determined by the investigator.
Be receiving valganciclovir, ganciclovir, cidofovir, foscarnet, leflunomide, letermovir, or artesunate when study treatment is initiated, or anticipated to require one of these agents during the 8-week treatment period.
Have a known hypersensitivity to maribavir or to any excipients.
Have severe vomiting, diarrhea, or other severe gastrointestinal (GI) illness within 24 hours prior to the first dose of study treatment or a GI absorption abnormality that would preclude administration of oral medication.
Require mechanical ventilation or vasopressors for hemodynamic support at baseline (Week 0).
Be pregnant (or expecting to conceive) or nursing.
Have previously completed, discontinued, or have been withdrawn from this study.
Have received any investigational agent or device within 30 days before initiation of study treatment (includes CMV specific T-cells). Previously approved agents under investigation for additional indications are not exclusionary.
Have previously received maribavir or CMV vaccine at any time.
Have any clinically significant medical or surgical condition that, in the investigator's opinion, could interfere with interpretation of study results, contraindicate the administration of the study treatment, or compromise the safety or well-being of the participant.
Have severe liver disease (Child-Pugh score of >=10).
Have serum aspartate aminotransferase greater than (>) 5 times upper limit of normal (ULN) at screening, or serum alanine aminotransferase>5 times ULN at screening, or total bilirubin>=3.0 times ULN at screening (except for documented Gilbert's syndrome), as analyzed by local laboratory.
Have positive results for human immunodeficiency virus (HIV). Participants must have a confirmed negative HIV test result within 3 months of study entry or, if unavailable, be tested by a local laboratory during the screening period.
Have active malignancy with the exception of nonmelanoma skin cancer, as determined by the investigator. Participants who experience relapse or progression of their underlying malignancy (for which HSCT or SOT was performed), as determined by the investigator, are not to be enrolled.
Be undergoing treatment for acute or chronic hepatitis B or hepatitis C.
Requiring ongoing treatment with or an anticipated need for treatment with a strong cytochrome P450 3A (CYP3A) inducer.
Have a low body weight where total blood volume (TBV) required during study participation will exceed 1 percent (%) TBV per study visit or 3% TBV over 30-day period.

### Statistical Methodology:

All analyses will be descriptive.

Internal interim analysis of safety and PK data will be conducted in at least 5 subjects of each cohort who complete Week 1 (Day 7 [+2 days]) PK sample collection. The objective of this interim analysis is to confirm the dose selection, to allow for dose adjustment, and to monitor the safety and tolerability of maribavir within each cohort.

An additional interim analysis will be conducted after 33 asymptomatic HSCT subjects over the 3 cohorts complete the study (with at least 10 asymptomatic HSCT subjects each in Cohorts 1 and 2, and 13 asymptomatic HSCT subjects in Cohort 3 [including 3 asymptomatic HSCT subjects 0 years to <2 years of age]) to allow submission of data to regulatory agencies.

### Safety Assessment

Safety and tolerability of maribavir for the treatment of CMV infection in the pediatric population (children and adolescents) after HSCT or SOT will be assessed by evaluation of SAEs, AEs (including instances of CMV disease or new episodes of CMV infection), AEs causing discontinuation from maribavir treatment or from the study, AEs leading to death, and AEs of special interest (AESIs). Use of concomitant medications, clinical laboratory tests, vital signs, ECGs, abnormal physical findings, and immunosuppressant drug levels will be also summarized.

Two observation periods are defined for the purpose of safety analyses:
On treatment period: from the time of maribavir initiation through 7 days after the last dose of study treatment
Overall study period: start of maribavir administration through the end of the study.

The safety endpoints will be summarized descriptively for the on-treatment period, and the overall study period, as appropriate. Baseline (Visit 2/Day 1/Week 1) assessments will be the last assessment before the first dose of maribavir treatment.

A treatment-emergent AE (TEAE) is defined as any event emerging or manifesting at or after the initiation of treatment with an investigational product or medicinal product or any existing event that worsens in either intensity or frequency following exposure to the investigational product or medicinal product.

The number of events, incidence, and percentage of TEAEs will be displayed for each cohort and overall by preferred terms (PTs) using the Medical Dictionary for Regulatory Activities for the on-treatment period and overall study period. Summaries in terms of severity and relationship to maribavir treatment will also be provided. Treatment-emergent SAEs will be summarized separately in a similar fashion. Summaries of AEs causing discontinuation of maribavir treatment, withdrawals, AEs leading to death, SAEs, and AESIs will be provided.

Adverse events of special interest will be analyzed according to primary system organ classes (SOCs) and PTs. Summary tables with SOCs and PTs will be generated presenting the number and percentage of subjects by AE, severity, seriousness, and relationship to maribavir treatment for the on-treatment period and overall study period. Invasive or opportunistic bacterial, viral and fungal infections, if any, will be noted.

Use of concomitant medications will be summarized descriptively for each of the cohorts and overall for the on treatment period and overall study period. Additionally, administration of hematopoietic growth factors, blood, and blood products will be summarized.

Change from baseline (Visit 2/Day 1/Week 1) in vital signs and clinical laboratory tests will be summarized for each cohort and overall with descriptive statistics at each assessment visit. Potentially clinically important findings will also be summarized.

Maribavir dose interruptions for any AE will be summarized. A summary of ECG findings will be provided by cohort and overall.

### Pharmacokinetic Analysis

An internal interim PK analysis will be conducted in at least 5 subjects of each cohort who complete Week 1 (Day 7 [+2 days]) PK sample collection. For interim PK analyses, noncompartmental analysis (NCA) will be performed on the total plasma concentration(s) of maribavir to characterize AUC, Cmax, and Cmin of each subject with serial PK sampling at Week 1. The target PK exposure for efficacy is a group geometric mean AUC within the range of 142 to 406 h*μg/mL and Cmin of ≥5.43 μg/mL.

Maribavir concentrations will be summarized by visit and planned sampling time. A scatterplot of all reportable maribavir concentration versus actual sampling time will be generated. A listing of subjects with maribavir concentration below the quantitation limit will be provided. The relationship between maribavir PK parameters (such as CL/F, Vz/F, λz, and half-life [t½]) and body weight or age may be explored if data allows.

For the subjects for which serial PK blood sampling will be conducted, the PK parameters will be calculated from maribavir concentration-time data using NCA and all calculations will be based on actual sampling times.

For all sparse PK sampling periods, no NCA will be conducted.

In a separate analysis and report, all maribavir concentrations obtained in this study (serial and sparse PK samples) will be combined with PK data from the Phase 2 and 3 studies in adults and analyzed by population PK analysis approach with a nonlinear mixed effect model approach using NONMEM Version 7 or above. Relationship between post hoc maribavir exposure from the population model and efficacy and safety endpoints of special interest may be explored if data allows.

### Efficacy Analysis

All efficacy endpoints will be summarized descriptively for each age cohort and overall. The denominator for the percentages will be based on the number of subjects in each age cohort and overall. Time-to-event endpoints will be summarized using Kaplan-Meier estimation; 95% confidence intervals for the estimated 25%, 50%, and 75% times will be presented.

### Palatability Assessment Analysis

Palatability data will be summarized descriptively for each age cohort and overall at Weeks 1, 4, and 8 (or end of treatment).

### Example 2: A Phase 1, Open-Label, Randomized, Two-Part Study in Healthy Adult Participants to Evaluate the Relative Bioavailability of Maribavir Pediatric Formulation Compared to the Commercial Formulation, as Well as, Food Effect, and Rabeprazole Gastric Acid-Reducing Effect on the Pharmacokinetics of Single-Dose Maribavir Pediatric Formulation

### Study Description

The study will have 2 parts, Part 1 and Part 2. Participants will only participate in one part.

The main aim of Part 1 of this study is to check the ability of a single dose of maribavir pediatric formulation to be absorbed in the digestive tract compared to commercial tablet formulation and to check how a high-fat, high-calorie meal affects absorption, distribution, and elimination of maribavir pediatric formulation given orally as water suspension.

The main aim of Part 2 of this study is to assess the stomach acid reducing effect of multiple doses of rabeprazole, on absorption, distribution, and elimination of maribavir pediatric formulation given orally as water suspension.

Each participant will stay in the study clinic from the day before the first treatment until the day after the last treatment.

Part 1 is a crossover design with three treatments (Treatments A, B, and C), six sequences, and three periods.

The relative bioavailability of 200 milligrams (mg) maribavir pediatric formulation administered orally as water suspension under fasting conditions (Treatment B) will be compared to 200 mg maribavir commercial tablet administered orally under fasting conditions (Treatment A). In addition, the effect of food on the pharmacokinetics (PK) of 200 mg maribavir pediatric formulation administered orally as water suspension under fasting conditions (Treatment B) and fed conditions (Treatment C) will be assessed. In each sequence, participants will receive three treatments (Treatments A, B, and C) per schedule.
Sequence 1: Treatment A+Treatment B+Treatment C
Sequence 2: Treatment A+Treatment C+Treatment B
Sequence 3: Treatment B+Treatment A+Treatment C.
Sequence 4: Treatment B+Treatment C+Treatment A
Sequence 5: Treatment C+Treatment A+Treatment B
Sequence 6: Treatment C+Treatment B+Treatment A

Part 2 is a single fixed-sequence design with two treatments (Treatments D and E). The two treatments will be administered to evaluate the gastric acid-reducing effect of multiple doses of rabeprazole on the PK of a single dose of 200 mg maribavir pediatric formulation administered orally as water suspension

| Arms | Assigned Interventions |
|---|---|
| Experimental: Part 1, Sequence 1: Treatment | Drug: Maribavir Commercial Tablet |
| A + Treatment B + Treatment C | Formulation |
| Participants will receive maribavir single | Maribavir commercial tablet. |
| 200 mg commercial tablet, on Day 1 of | Drug: Maribavir Pediatric Powder- |
| Treatment Period 1 under fasting | for-oral Suspension Formulation |
| condition (Treatment A), followed by | Maribavir pediatric powder-for- |
| maribavir 200 mg pediatric powder-for- | oral suspension. |
| oral suspension, single oral dose on Day 1 | |
| of Treatment Period 2 under fasting | |
| condition (Treatment B), and further | |
| followed by maribavir 200 mg pediatric | |
| powder-for-oral suspension, single oral | |
| dose on Day 1 of Treatment Period 3 | |
| administered with a high fat/high calorie | |
| meal (Treatment C). There will be a | |
| washout period of a minimum of 72 hours | |
| between each treatment. | |
| Experimental: Part 1, Sequence 2: Treatment | Drug: Maribavir Commercial Tablet |
| A + Treatment C + Treatment B | Formulation |
| Participants will receive single maribavir | Maribavir commercial tablet. |
| 200 mg commercial tablet, on Day 1 of | Drug: Maribavir Pediatric Powder- |
| Treatment Period 1 under fasting | for-oral Suspension Formulation |
| condition (Treatment A), followed by | Maribavir pediatric powder-for- |
| maribavir 200 mg pediatric powder-for- | oral suspension. |
| oral suspension, single oral dose on Day 1 | |
| of Treatment Period 2 administered with | |
| a high fat/high calorie meal (Treatment | |
| C), and further followed by maribavir 200 | |
| mg pediatric powder-for-oral suspension, | |
| single oral dose on Day 1 of Treatment | |
| Period 3 under fasting condition | |
| (Treatment B). There will be a washout | |
| period of a minimum of 72 hours between | |
| each treatment. | |
| Experimental: Part 1, Sequence 3: Treatment | Drug: Maribavir Commercial Tablet |
| B + Treatment A + Treatment C | Formulation |
| Participants will receive maribavir 200 | Maribavir commercial tablet. |
| mg pediatric powder-for-oral suspension, | Drug: Maribavir Pediatric Powder- |
| single oral dose on Day 1 of Treatment | for-oral Suspension Formulation |
| Period 1 under fasting condition | Maribavir pediatric powder-for- |
| (Treatment B), followed by maribavir | oral suspension. |
| single 200 mg commercial tablet, on Day | |
| 1 of Treatment Period 2 under fasting | |
| condition (Treatment A), and further | |
| followed by maribavir 200 mg pediatric | |
| powder-for-oral suspension, single oral | |
| dose on Day 1 of Treatment Period 3 | |
| administered with a high fat/high calorie | |
| meal (Treatment C). There will be a | |
| washout period of a minimum of 72 hours | |
| between each treatment. | |
| Experimental: Part 1, Sequence 4: Treatment | Drug: Maribavir Commercial Tablet |
| B + Treatment C + Treatment A | Formulation |
| Participants will receive maribavir 200 | Maribavir commercial tablet. |
| mg pediatric powder-for-oral suspension, | Drug: Maribavir Pediatric Powder- |
| single oral dose on Day 1 of Treatment | for-oral Suspension Formulation |
| Period 1 under fasting condition | Maribavir pediatric powder-for- |
| (Treatment B), followed by maribavir 200 | oral suspension. |
| mg pediatric powder-for-oral suspension, | |
| single oral dose on Day 1 of Treatment | |
| Period 2 administered with a high fat/high | |
| calorie meal (Treatment C), and further | |
| followed by maribavir single 200 mg | |
| commercial tablet, on Day 1 of Treatment | |
| Period 3 under fasting condition | |
| (Treatment A). There will be a washout | |
| period of a minimum of 72 hours between | |
| each treatment. | |
| Experimental: Part 1, Sequence 5: Treatment | Drug: Maribavir Commercial Tablet |
| C + Treatment A + Treatment B | Formulation |
| Participants will receive maribavir 200 | Maribavir commercial tablet. |
| mg pediatric powder-for-oral suspension, | Drug: Maribavir Pediatric Powder- |
| single oral dose on Day 1 of Treatment | for-oral Suspension Formulation |
| Period 1 administered with a high fat/high | Maribavir pediatric powder-for- |
| calorie meal (Treatment C), followed by | oral suspension. |
| maribavir single 200 mg commercial | |
| tablet, on Day 1 of Treatment Period 2 | |
| under fasting condition (Treatment A), | |
| and further followed by maribavir 200 mg | |
| pediatric powder-for-oral suspension, | |
| single oral dose on Day 1 of Treatment | |
| Period 3 under fasting condition | |
| (Treatment B). There will be a washout | |
| period of a minimum of 72 hours between | |
| each treatment. | |
| Experimental: Part 1, Sequence 6: Treatment | Drug: Maribavir Commercial Tablet |
| C + Treatment B + Treatment A | Formulation |
| Participants will receive maribavir 200 | Maribavir commercial tablet. |
| mg pediatric powder-for-oral suspension, | Drug: Maribavir Pediatric Powder- |
| single oral dose on Day 1 of Treatment | for-oral Suspension Formulation |
| Period 1 administered with a high fat/high | Maribavir pediatric powder-for- |
| calorie meal (Treatment C), followed by | oral suspension. |
| maribavir 200 mg pediatric powder-for- | |
| oral suspension, single oral dose on Day 1 | |
| of Treatment Period 2 under fasting | |
| condition (Treatment B), and further | |
| followed by maribavir single 200 mg | |
| commercial tablet, on Day 1 of Treatment | |
| Period 3 under fasting condition as | |
| (Treatment A). There will be a washout | |
| period of a minimum of 72 hours between | |
| each treatment. | |
| Experimental: Part 2: Treatment D: | Drug: Maribavir Pediatric Powder- |
| Maribavir 200 mg | for-oral Suspension Formulation |
| Participants will receive maribavir 200 | Maribavir pediatric powder-for- |
| mg pediatric powder-for-oral suspension, | oral suspension. |
| single oral dose on Day 1 of Treatment | |
| Period 1 under fasting condition | |
| (Treatment D). | |
| Experimental: Part 2, Treatment E: | Drug: Maribavir Pediatric Powder- |
| Rabeprazole 20 mg + maribavir 200 mg | for-oral Suspension Formulation |
| Participants will receive rabeprazole | Maribavir pediatric powder-for- |
| single 20 mg tablet, once daily on Days 1 | oral suspension. |
| to 5 of Treatment Period 2 under fasting | Drug: Rabeprazole |
| condition followed by maribavir 200 mg | Rabeprazole tablet. |
| pediatric powder-for-oral suspension, | |
| single oral dose, 2 hours after rabeprazole | |
| dosing on morning of Day 5 of Treatment | |
| Period 2 under fasting condition | |
| (Treatment E). There will be a washout | |
| period of a minimum of 72 hours between | |
| maribavir dosing in Treatment Period 1 | |
| and first dose of rabeprazole in Treatment | |
| Period 2. | |

### Outcome Measurements

### Primary Outcome Measure:

### 1. Parts 1 and 2: Maximum Observed Plasma Concentration (Cmax) of Maribavir

Cmax of maribavir in plasma will be assessed.

[Time Frame: Pre-dose, 0.25, 0.5, 1, 1.5, 2, 3, 4, 5, 6, 8, 12, 16 and 24 hours post-dose in each treatment period on Day 1 in Parts 1 and 2 and Day 5 in Part 2]

2. Parts 1 and 2: Area Under the Plasma Concentration-Time Curve from Time 0 to the Time of the Last Quantifiable Concentration (AUClast) of Maribavir

AUClast of maribavir in plasma will be assessed.

[Time Frame: Pre-dose, 0.25, 0.5, 1, 1.5, 2, 3, 4, 5, 6, 8, 12, 16 and 24 hours post-dose in each treatment period on Day 1 in Parts 1 and 2 and Day 5 in Part 2]

3. Parts 1 and 2: Area Under the Plasma Concentration-Time Curve from Time 0 to Infinity (AUCO-Infinity) of Maribavir

AUCO-infinity of maribavir in plasma will be assessed.

[Time Frame: Pre-dose, 0.25, 0.5, 1, 1.5, 2, 3, 4, 5, 6, 8, 12, 16 and 24 hours post-dose in each treatment period on Day 1 in Parts 1 and 2 and Day 5 in Part 2]

### Secondary Outcome Measure:

4. Parts 1 and 2: Number of Participants with Treatment-Emergent Adverse Events (TEAEs) and Serious Adverse Events (SAEs)

TEAEs will include TEAES by severity, TEAEs by causality, any clinically significant changes in vital signs, electrocardiogram (ECG) values and clinical laboratory parameters.

[Time Frame: Parts 1 and 2: From start of study drug administration up to follow-up (Day 16).

### Eligibility

Minimum Age: 18 Years
Maximum Age: 55 Years
Sex: All
Gender Based: No
Accepts Healthy Volunteers: Yes

### Inclusion Criteria

An understanding, ability, and willingness to fully comply with study procedures and restrictions and to voluntarily sign (personally or via a legally authorized representative) informed consent form to participate in the study.

Age 18 to 55 years, inclusive at the time of consent, at the screening visit.

Male, or non-pregnant, non-breastfeeding female who agrees to comply with any applicable contraceptive requirements of the protocol or female of non-childbearing potential.

Body mass index (BMI) between 18.0 and 30.0 kilogram per meter square (kg/m\hat{ }2), inclusive with a body weight greater than (>) 50 kilograms (kg) (110 pounds [lbs]), at the screening visit.

Healthy as determined by the Investigator or designee on the basis of screening evaluations and medical history.

Hemoglobin for males greater than or equal to (>=) 135.0 gram per liter (g/L) and females>=120.0 g/L, at the screening visit and on Day-1 of Treatment Period 1.

Ability to swallow a dose of maribavir or rabeprazole.

### Exclusion Criteria

History or presence of gastritis, gastrointestinal (GI) tract disorder, hepatic disorder or cholecystectomy, history of treated or untreated Helicobacter pylori, ulcer disease or other clinical condition which, in the opinion of the investigator or designee, may affect the absorption, distribution, metabolism, or elimination of the study drugs.

History of any hematological, hepatic, respiratory, cardiovascular, renal, neurological or psychiatric disease, gall bladder removal, or current recurrent disease that could affect the action, absorption, or disposition of the study drugs, or clinical or laboratory assessments.

Current or relevant history of physical or psychiatric illness, any medical disorder that may require treatment or make the participant unlikely to fully complete the study, or any condition that presents undue risk from the study drugs or procedures.

Known or suspected intolerance or hypersensitivity to maribavir or rabeprazole (Part 2 only), closely related compounds, or any of the stated ingredients and excipients.

Significant illness, as judged by the Investigator or designee, within 2 weeks of the first dose of the investigational drug (ID).

Has diarrhea within 4 hours of the first dose of the ID.

Donation of blood or blood products (example, plasma or platelets) within 60 days prior to receiving the first dose of the ID.

Within 30 days prior to the first dose of the ID:

Have used any investigational product (if elimination half-life is less than [<] 6 days, otherwise 5 half-lives).

Have been enrolled in a clinical study (including vaccine studies) that, in the Investigator or designee's opinion, may impact this Takeda-sponsored study.

Have had any substantial changes in eating habits, as assessed by the Investigator or designee.

Systolic blood pressure>140 millimeters of mercury (mmHg) or <90 mmHg, and/or diastolic blood pressure>90 mmHg or <50 mmHg, at the screening visit.

Corrected QT interval (QTc)>450 millisecond (msec) at the screening visit. If QTc exceeds 450 msec, the ECG should be repeated two more times and the average of the three QTc values should be used to determine the participant's eligibility.

Known history of alcohol or other substance abuse within the last year.

Male participants who consume more than 21 units of alcohol per week or three units per day. Female participants who consume more than 14 units of alcohol per week or two units per day (one alcohol unit=one beer or one wine [5 ounces [oz]/150 milliliter [mL]] or one liquor [1.5 oz/40 mL] or 0.75 oz alcohol).

A positive screen for alcohol or drugs of abuse at the screening visit or on Day −1 of Treatment Period 1. Urine samples are to be tested for amphetamines, barbiturates, benzodiazepines, cannabinoids, cocaine, methadone, opiates, and phencyclidine.

A positive Human immunodeficiency virus (HIV), Hepatitis B surface antigen (HBsAg), or Hepatitis C virus (HCV) antibody screen at the screening visit.

Use of tobacco in any form (example, smoking or chewing) or other nicotine-containing products in any form (example, gum, patch). Ex-users must self-report that they have stopped using tobacco for at least 3 months prior to receiving the first dose.

Routine consumption of more than two units of caffeine per day or participants who experience caffeine withdrawal headaches (One caffeine unit is contained in the following items: one 6-oz [180 mL] cup of coffee, two 12-oz [360 mL] cans of cola, one 12-oz cup of tea, three 1-oz [85 grams [g]] chocolate bars). Decaffeinated coffee, tea, or cola are not considered to contain caffeine.

Current use of any prescription medication with the exception of hormonal contraceptives and hormonal replacement therapy. Current use of any over-the-counter (OTC) medication (including OTC multi-vitamin, herbal, or homeopathic preparations) within 14 days of the first dose. Hormonal contraceptives and hormonal replacement therapy may be permitted if the female participant has been on the same stable dose for at least 3 months prior to first dose.

Current use of antacids, proton pump inhibitors (PPIs), or histamine type 2 (H2) antagonists within 14 days of the first dose, except for on-study rabeprazole.

Inability or unwillingness to consume 100 percent of the high-fat, high-calorie meal (including participants with lactose or gluten intolerance).

Female participants with a positive pregnancy test at the screening visit or on Day −1 of Treatment Period 1 or who are lactating.

Participants on a diet incompatible with the on-study diet, in the opinion of the Investigator or designee, within the 30 days prior to the first dosing and throughout the study.

Recent history (within 1 month) of oral/nasal cavity infections, history of gastroesophageal reflux, asthma treatment with albuterol, or zinc supplementation.

Participants with dry mouth syndrome or burning mouth syndrome or participants suffering from dysgeusia.

### Results

A total of 18 participants entered the study into Part 1 and were randomized to 1 of 6 treatment sequences (ABC, ACB, BAC, BCA, CAB, or CBA). All 18 participants completed the study.

A total of 14 participants entered the study into Part 2. All 14 participants completed the study.

Pharmacokinetics: Plasma maribavir Cmax following a single oral dose of maribavir powder-for-oral-suspension formulation administered as a water suspension under fasting conditions (Treatment B) was approximately 18% lower than that following a single oral 200 mg dose of maribavir administered as the commercial tablet under fasting conditions (Treatment A), while the plasma maribavir AUClast and AUC∞ were similar following both treatments. The results of nonparametric analysis also suggested that tmax and tlag of plasma maribavir were not statistically different following Treatment A as compared to Treatment B. Pharmacokinetic data is shown in the tables below:

TABLE
Pharmacokinetic Assessments

| | Treatment | | | | | |
|---|---|---|---|---|---|---|
| | Single oral dose 200-mg maribavir | | | | | |
| | Treatment A | | Treatment B | | Treatment C | |
| | Commercial | | Powder-for-oral | | Powder-for-oral | |
| | tablet under | | suspension under | | suspension following high- | |
| | fasting conditions | | fasting conditions | | fat/high-calorie meal | |
| Pharmacokinetic parameters | (n = 18) | | (n = 18) | | (n = 18) | |
| tₘₐₓ, median (range), hr | 1.00 | (0.50-3.00) | 1.25 | (0.99-2.03) | 4.00 | (2.00-8.00) |
| Cₘₐₓ, μg/mL | | | | | | |
| Mean ± SD | 13.0 ± 3.5 | | 10.7 ± 2.82 | | 6.21 ± 1.81 | |
| GM (GCV) | 12.6 | (26.3) | 10.4 | (26.2) | 5.98 | (28.3) |
| C_{12 h}, μg/mL | | | | | | |
| Mean ± SD | 1.29 ± 1.11 | | 1.32 ± 0.871 | | 1.62 ± 1.17 | |
| GM (GCV) | — | | — | | 1.26 | (90.8) |
| AUC_{12 h}, μg · hr/mL | | | | | | |
| Mean ± SD | 54.3 ± 25.4 | | 50.6 ± 19.2 | | 39.5 ± 16.3 | |
| GM (GCV) | 49.7 | (44.3) | 47.2 | (40.7) | 36.7 | (39.9) |
| AUCₗₐₛₜ, GM (GCV), μg · hr/mL | | | | | | |
| Mean ± SD | 62.6 ± 34.2 | | 59.9 ± 27.5 | | 49.9 ± 26.7 | |
| GM (GCV) | 55.5 | (52.6) | 54.2 | (50.2) | 44.4 | (51.9) |
| AUC_{∞}, μg · hr/mL | | | | | | |
| Mean ± SD | 67.6 ± 39.2 | | 67.4 ± 38.5 | | 55.9 ± 36.5 | |
| GM (GCV) | 59.1 | (55.9) | 59.1 | (56.3) | 48.2 | (57.6) |
| AUCₑₓₜᵣₐₚ %, % | | | | | | |
| Mean ± SD | 6.02 ± 3.55 | | 8.00 ± 7.14 | | 7.72 ± 5.39 | |
| GM (GCV) | 5.23 | (56.9) | 6.12 | (82.0) | 6.76 | (50.1) |
| t_{lag}, median (range), hr | 0.00 | (0.00-0.26) | 0.00 | (0.00-0.25) | 0.25 | (0.00-0.55) |
| t_{1/2z}, mean ± SD, hr | 5.38 ± 2.53 | | 6.71 ± 3.76 | | 5.53 ± 2.45 | |
| λ_{z}, mean ± SD, 1/hr | 0.164 ± 0.0900 | | 0.134 ± 0.0740 | | 0.149 ± 0.0655 | |
| CL/F, mean ± SD, L/hr | 3.82 ± 1.94 | | 3.85 ± 2.13 | | 4.69 ± 2.29 | |
| V_{z}/F, mean ± SD, L | 24.2 ± 6.34 | | 29.6 ± 6.78 | | 30.9 ± 6.34 | |
| C_{τ}, μg/mL | | | | | | |

| Mean ± SD | 1.89 ± 1.86 | 2.17 ± 2.13 | 2.36 ± 2.36 | |
|---|---|---|---|---|
| GM (GCV) | — | — | 1.62 | (117) |

TABLE
Pharmacokinetic/Bioavailability Assessments

| | Treatment | | | | | | |
|---|---|---|---|---|---|---|---|
| | Single oral dose 200 mg maribavir | | | | | | |
| | Treatment B | | Treatment A | | | | |
| | Under fasting | | Under fasting | | | | |
| | conditions | | conditions | | | | |
| | (Test) | | (Reference) | | | | |
| PK | | Geometric | | Geometric | GMR | | Intraparticipant |
| parameter | n | LSMs | n | LSMs | (%) | 90% CI | CV % |
| Cₘₐₓ, | 18 | 10.4 | 18 | 12.6 | 82.19 | 74.31-90.91 | 18.00 |
| μg/mL | | | | | | | |
| AUCₗₐₛₜ, | 18 | 54.2 | 18 | 55.5 | 97.59 | 91.39-104.20 | 11.65 |
| μg · h/mL | | | | | | | |
| AUC_{∞}, | 18 | 59.1 | 18 | 59.1 | 99.94 | 94.12-106.11 | 10.65 |
| μg · h/mL | | | | | | | |

| | Single oral dose 200 mg maribavir | | | | | | |
|---|---|---|---|---|---|---|---|
| | Treatment C | | Treatment A | | | | |
| | Following high-fat/ | | Under fasting | | | | |
| | high-calorie meal | | conditions | | | | |
| | (Test) | | (Reference) | | | | |
| PK | | Geometric | | Geometric | GMR | | Intraparticipant |
| parameter | n | LSMs | n | LSMs | (%) | 90% CI | CV % |
| Cₘₐₓ, | 18 | 5.98 | 18 | 10.4 | 57.72 | 52.18-63.84 | 18.00 |
| μg/mL | | | | | | | |
| AUCₗₐₛₜ, | 18 | 44.4 | 18 | 54.2 | 82.05 | 76.84-87.61 | 11.65 |
| μg · h/mL | | | | | | | |
| AUC_{∞}, | 18 | 48.2 | 18 | 59.1 | 81.68 | 76.93-86.73 | 10.65 |
| μg · h/mL | | | | | | | |

Palatability Assessment: All 18 participants in Part 1 assessed a single oral 200 mg dose of maribavir powder-for-oral suspension formulation administered as a water suspension under fasted conditions (Treatment B) and a single oral 200 mg dose of maribavir powder-for-oral suspension formulation administered as a water suspension under fed conditions (Treatment C) as easy to swallow. For Treatment B, the majority (72%) agreed the overall taste and texture of the drug was acceptable, the drug did not have a rough or gritty texture (72%), and the medication smell was neither good nor bad (67%). The participants assessed Treatment B as sweet (61%), bitter (33%), or bitter/sweet (6%) and the majority assessed the taste as medium (44%) or strong (44%). The most common drug taste and strength combination was sweet, medium (39%), bitter/strong (28%) and sweet/strong (17%). For Treatment C, the majority (83%) agreed the overall taste and texture of the drug was acceptable, the drug did not have a rough or gritty texture (67%), and the medication smell was neither good nor bad (61%). The participants assessed Treatment C as bitter (44%), sweet (39%), bitter/sweet (11%) and savory (6%) and the majority assessed the taste as medium (50%) or strong (44%). The most common drug taste and strength combination was sweet, medium (33%), bitter/strong (28%), and bitter, medium (17%). The palatability assessment between a single oral 200 mg dose of maribavir powder-for-oral suspension formulation administered as a water suspension under fasted conditions (Treatment B) and under fed conditions (Treatment C) was comparable, with the exception of how sweet the drug tasted. 11 (61%) participants assessed Treatment B as sweet versus 7 (39%) of participants for Treatment C. Palatability data is shown in the table below:

TABLE
Palatability Assessment

| | | n (%) | | | |
|---|---|---|---|---|---|
| | | Treatment B | | Treatment C | |
| Question | Response | (n = 18) | | (n = 18) | |
| How did this drug taste to you? | Bitter | 6 | (33) | 8 | (44) |
| | Bitter/Sweet | 1 | (6) | 2 | (11) |
| | Savory | 0 | (0) | 1 | (6) |
| | Sweet | 11 | (61) | 7 | (39) |
| How strong was the taste? | Medium | 8 | (44) | 9 | (50) |
| | Strong | 8 | (44) | 8 | (44) |
| | Weak | 2 | (11) | 1 | (6) |
| How did the drug taste and how | Bitter, Medium | 1 | (6) | 3 | (17) |
| strong? | Bitter, Strong | 5 | (28) | 5 | (28) |
| | Bitter/Sweet, Strong | 0 | (0) | 2 | (11) |
| | Bitter/Sweet, Weak | 1 | (6) | 0 | (0) |
| | Savory, Weak | 0 | (0) | 1 | (6) |
| | Sweet, Medium | 7 | (39) | 6 | (33) |
| | Sweet, Strong | 3 | (17) | 1 | (6) |
| | Sweet, Weak | 1 | (6) | 0 | (0) |
| Did drug have a rough or | No | 13 | (72) | 12 | (67) |
| gritty texture? | Yes | 5 | (28) | 6 | (33) |
| Was the drug easy to swallow? | Yes | 18 | (100) | 18 | (100) |
| The overall taste and texture of | Agree | 13 | (72) | 15 | (83) |
| the drug was acceptable. | Disagree | 2 | (11) | 2 | (11) |
| | Neither Agree Nor Disagree | 3 | (17) | 1 | (6) |
| How does the medication smell | Good | 6 | (33) | 7 | (39) |
| to you? | Neither Good Nor Bad | 12 | (67) | 11 | (61) |

### Example 3: Preparation of Maribavir Formulation

Unsieved maribavir pellets (65% w/w): VG-25 granulator was turned on and adjusted to a chopper speed of 1,500 rpm, a main impeller speed of 150 rpm, and atomizing air pressure of 1.5 bar. The pump and tubing were primed and calibrated with purified water, USP (500 g). Micronized maribavir (1,300 g), Avicel PH-105® (Microcrystalline cellulose, NF, Ph. Eur, JP; 620.0 g), and Polyplasdone™ XL-10 Crospovidone (Type B NF, EP, JP; 80.0 g) were added to the VG-25 granulator in the recited order, and were blended for ˜1 minute. The VG-25 granulator was prepared for spraying by spraying purified water USP (200 g) at 25 g/min for about 8 minutes, and any remaining water was discarded. The pre-wetted blend was then discharged into a double poly-lined container and the weight recorded (1,941.6 g).

The pre-wetted blend was divided into two equal portions, each 970.8 g. The pump and tubing were primed and calibrated with purified water, USP to ensure that the pump can deliver at rates of 15 and 40 g/min. The first portion of the pre-wetted blend was loaded onto the powder feeder, and the powder feeder was run for 60 seconds to verify that it could deliver at a rate between 10-20 g/min, and the collected material was returned back to the powder feeder. The powder feeder was ran as shown in the table below, and the median particle size was monitored periodically using an Eyecon Particle Size Analyzer:

| | | | | |
|---|---|---|---|---|
| Process | Air Volume | Spray Rate | Rotor Speed | |
| Adjustment | 60-140 | 15-40 | 950-1050 | |
| Points | m³/hr | g/min | RPM | Notes |

Pelletizing

| | | | | |
|---|---|---|---|---|
| Start | 80 | 40 | 1,000 | Start Process and |
| | | | | Spraying |
| ~300 g sprayed | | 20 | | Reduce Spray Rate |
| Medium pellet | | 15 | | Reduce Spray Rate |
| size of ~200- | | | | |
| 300 μm by Eyecon | | | | |
| Medium pellet | | Stop Spray | | Engage Powder |
| size of ~250- | | | | Feeder - 15 g/min |
| 350 μm by Eyecon | | | | Apply the dry powder |
| Powder in Feeder | | | | Continue tumbling |
| Depleted Continue | | | | for 3-5 minutes |
| tumbling for | | | | |
| 3-5 minutes | | | | |

Stop the pelletization process
Closing the rotor knob (moving rotor plate to the lowest position)
Turn off the rotor
Turn of the fluidization air

The wet pellets were discharged into a poly-lined container and the weight was recorded as 1,217.8 g. The second portion was then processed using the same parameters described above, and was weighted (1,204.9 g).

The wet pellets were the dried by first pre-heating and charging both portions of the wet pellets into the fluid bed with a targeted product temperature of 57° C.±5° C. Once the targeted product temperature was achieved, a sample was removed to confirmed it was dried to <2.0% loss on drying “LOD”. If LOD is not <2.0%, drying is repeated.

Once LOD was met, the pellets were discharged into double polyethylene lined 5-gallon vessel(s) (1,852.2 g, 84.2% yield). A portion of the unsieved maribavir pellets 65% w/w (20.0 g) was removed for in-process testing. For example, the density was determined to be 0.69 g/mL. In addition, the particle size was analyzed as shown in the table below:

| | | Qty. | % Retained |
|---|---|---|---|
| Screen | Opening | Retained | (Wt. Retained + |
| (US Mesh) | (μm) | (g) | Total) × 100 |
| 40 | 425 | 1.1 | 5.6 |
| 45 | 355 | 0.6 | 3.1 |
| 50 | 300 | 1.2 | 6.2 |
| 60 | 250 | 4.3 | 22.1 |
| 70 | 212 | 10.1 | 51.8 |
| 80 | 180 | 1.4 | 7.2 |
| Pan | <180 | 0.8 | 4.1 |

| | | |
|---|---|---|
| Total: | 19.5 | 100.1 |

Preparation of sieved maribavir pellets (65% w/w). Sieved maribavir pellets (65% w/w) were produced from unsieved pellets (65% w/w) through use of U.S. Standard #45 Mesh Hand Screen and U.S. Standard #80 Mesh Hand Screen. Acceptable pellets were those that i) passed through the U.S. Standard #45 Mesh Hand Screen and ii) retained on top of the U.S. Standard #80 Mesh Hand Screen.

Preparation of coated maribavir pellets (36.1% w/w). Purified water, USP (3,840.0 g) was dispensed into a container with a mixer and blade. While stirring the purified water with a propeller mixer to form a vortex, Eudragit® E PO ReadyMix (960.0 g) was added, and the mixture was mixed for 15 minutes, after which the speed was reduced and mixing contained for an additional 45 minutes prior to starting coating. The net weight of the coating suspension was determined to be 4,783.5 g, which was within ±3.0% of the theoretical net weight of 4,800.0 g.

The adjusted amount of coating suspension to be sprayed was calculated as:

$$[ Total Solid Content = 20 \% w / w ; Coated Weight Gain = 80 \% ] \\ sieved maribavir pellets 65 \% w / w ( 719.7 g ) \times 4 = 2 , 878 , 8 g$$

The pump and tubing were primed and calibrated with the coating suspension. In addition, the equipment was warmed and preconditioned for at least 10 minutes. The sieved maribavir pellets 65% w/w (719.7 g) were loaded into the fluid bed and coating was performed as shown below:

| | | | | | |
|---|---|---|---|---|---|
| | | | | Product | |
| Process Time | Air Volume | Spray Rate | Atomizing Air | Temperature | |
| (min) | 10-50 CFM | 5-25 g/min | 1.5-3.5 bar | 15-45° C. | Notes |

Coating Suspension

| | | | | | |
|---|---|---|---|---|---|
| 0 | 35 | 0 | 1.0 | Heating | Heat to minimum 36° C. |
| | | | | | product temp. |
| 1 | | 8 | 2.5 | Minimum | Start spray |
| | | | | 30° C. | |
| 15 | | 10 | | 30° C. | Spray rate increase |
| 30 | | 12 | | | Spray rate increase |
| 45 | | 14 | | | Spray rate increase |
| 60 | | 16 | | | Spray rate increase |
| 75-End Spray | | 18 | | | Spray amount: 2,878.8 g |

Purge

| | | |
|---|---|---|
| Start-End | 35 | Set Point |
| Spray | | 2.5 |

Drying

| | | | | |
|---|---|---|---|---|
| Until Product | 35 | 1.0 | Inlet Temp. | Atomizing air decrease |
| Temp. of 37° | | | Set Point 50° | |
| C. ± 5° C. | | | C. ± 10° C. | |

Cooling

| | | | |
|---|---|---|---|
| 5 min | 35 | Set Point | No Heat |
| (Range 3-10) | | 1.0 | (Cooling) |

End Process

The pellets were removed and sieved through a U.S. Standard #35 Mesh Hand screen, where any pellets retained on the mesh hand screen were discarded as over-sized. The remaining pellets are coated maribavir pellets 36% w/w (1,165.6 g, 90.0% yield).

A portion of the coated maribavir pellets 36% w/w (20.0 g) was removed for in-process testing. For example, the density was determined to be 0.74 g/mL. In addition, the particle size was analyzed as shown in the table below:

| | | Qty. | % Retained |
|---|---|---|---|
| Screen | Opening | Retained | (Wt. Retained + |
| (US Mesh) | (μm) | (g) | Total) × 100 |
| 40 | 425 | 0.0 | |
| 45 | 355 | 1.0 | 5.0 |
| 50 | 300 | 4.6 | 22.9 |
| 60 | 250 | 9.2 | 45.8 |
| 70 | 212 | 4.4 | 21.9 |
| 80 | 180 | 0.7 | 3.5 |
| Pan | <180 | 0.2 | 1.0 |

| | | |
|---|---|---|
| Total: | 20.1 | 100.1 |

### Example 4: Evaluation of Dissolution of Maribavir Under Specified Conditions

Preparation of 25 mM phosphate buffer with 0.2% TEA (pH 3.0): potassium phosphate monobasic (3.4 g) was weighed and transferred into a suitable container with 1,000 mL of USP water. The mixture was dissolved by mixing. Triethylamine (2.0 mL) was added and mixed well. The pH of the solution was adjusted to 3.0±0.05 with phosphoric acid (85%).

Preparation of mobile phase: A 25 mM phosphate buffer (pH 3.0, 700 mL) was combined with 300 mL of acetonitrile. The solution was mixed well and filtered through a 0.45-μm nylon membrane filter and degassed.

Preparation of diluent: dissolution medium (50 mM phosphate buffer at pH 2.5) was used as a diluent. A portion of the dissolution medium was filtered, and the first 5 mL of the filtrate was discarded. The clear filtrate was transferred into HPLC vials for dissolution medium blank analysis.

Maribavir working standard solution preparation: Maribavir reference standard (20 mg) was transferred to a 200-mL volumetric flask. Diluent (150 mL) was added to the flask and sonicated for about 10 minutes or until dissolved. The solution was brought to room temperature and diluted to volume with diluent. The concentration of maribavir in this standard solution was 0.1 mg/mL.

Dissolution media preparation (50 mM phosphate buffer at pH 2.5); Potassium monophosphate monobasic (68 mg) was weighed and dissolved in deionized water up to 10 L and mixed. The pH was adjusted to 2.5±0.05 with phosphoric acid solution, and was degassed.

Dissolution procedure: The dissolution apparatus (USP Apparatus II, Paddles) was assembled and the paddle was position so that the distance between its lower edge and the lower inner surface of the vessel was within 25±2 mm throughout the test. This was done for all six positions. The speed was set to 75 rpm, and the temperature was set to 37±0.05° C. Phosphate buffer pH 2.5 (500 mL) was placed in each of six vessels.

Maribavir (50 mg) (e.g., produced as described in Example 3) was weighed and placed into each of the six separate beakers, the weight was recorded, and the beads were transferred in each of the vessels, respectively. The paddle assembly was lowered to its lower most position, and the paddles were rotated at 75 rpm. Samples were withdrawn at 5, 10, 15, 20, 30, and 45 minute time points, after with the paddle rotation speed was increased to 250 rpm and ran for an additional 15 minutes. The samples were filtered (first 5 mL discarded) and transferred into HPLC vials. The samples were evaluated by HPLC against the standard solutions, and % dissolution was calculated as shown in FIG. 2 for each of four batches of maribavir.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. (canceled)

2. A pharmaceutical composition comprising:
(i) an intragranular composition comprising maribavir, a diluent, and disintegrant;
(ii) an enteric coating of the intragranular composition comprising a selectively soluble polymer; and
(iii) an extragranular composition comprising a diluent, a suspending agent, a sweetener, and flavorant.

3. (canceled)

4. The pharmaceutical composition of claim 2, comprising:
(i) an intragranular composition comprising maribavir, microcrystalline cellulose, and crospovidone;
(ii) an enteric coating of the intragranular composition comprising a selectively soluble polymer; and
(iii) an extragranular composition comprising a diluent, a suspending agent, a sweetener, and flavorant.

5. (canceled)

6. (canceled)

7. The pharmaceutical composition of claim 2, comprising:
(i) an intragranular composition comprising maribavir, microcrystalline cellulose, and crospovidone;
(ii) an enteric coating of the intragranular composition comprising a selectively soluble polymer; and
(iii) an extragranular composition comprising mannitol, a suspending agent, a sweetener, and flavorant.

8. (canceled)

9. (canceled)

10. The pharmaceutical composition of claim 2, comprising:
(i) an intragranular composition comprising maribavir, microcrystalline cellulose, and crospovidone;
(ii) an enteric coating of the intragranular composition comprising a selectively soluble polymer; and
(iii) an extragranular composition comprising mannitol, microcrystalline cellulose and carboxymethylcellulose sodium, sucralose, and flavorant.

11. The pharmaceutical composition of claim 2, further comprising a suspending agent selected from the group consisting of microcrystalline cellulose and carboxymethylcellulose sodium, or a combination thereof.

12. (canceled)

13. The pharmaceutical composition of claim 2, wherein the intragranular composition is composed of granules comprising maribavir, microcrystalline cellulose, and crospovidone.

14. The pharmaceutical composition of claim 2, wherein selectively soluble polymer is insoluble at neutral pH but soluble at acidic pH.

15. The pharmaceutical composition of claim 2, wherein the selectively soluble polymer is a butyl methylacrylate, dimethylaminoethyl methacrylate, methyl methacrylate copolymer (e.g., Eudragit® E PO).

16. The pharmaceutical composition of claim 2, wherein the sweetener is sucralose.

17. The pharmaceutical composition of claim 2, wherein the diluent is mannitol.

18. The pharmaceutical composition of claim 2, wherein the flavorant is a fruit flavor.

19. The pharmaceutical composition of claim 2, wherein the coated intragranular composition comprises granules having a median particle diameter of 225-350 microns.

20. The pharmaceutical composition of claim 2, wherein the coated intragranular composition comprises granules having a median particle diameter of about 250-325 microns, about 250-310 microns, about 250-300 microns, about 250-290 microns, about 250 280 microns, about 250-270 microns, or about 225-300 microns.

21. An oral liquid formulation of maribavir comprising the pharmaceutical composition of claim 2.

22. The oral liquid formulation of claim 21, wherein the pharmaceutical composition forms a suspension in water.

23. A method of treating cytomegalovirus (CMV) infection or disease in a patient suffering therefrom comprising administering to the patient a therapeutically effective amount of the pharmaceutical composition of claim 2.

24. The method of claim 23, wherein the pharmaceutical composition is in an oral liquid formulation of claim 22.

25. (canceled)

26. The method of claim 23, wherein the patient has a CMV deoxyribonucleic acid (DNA) screening value of ≥1365 International Units per milliliter (IU/mL) in whole blood or ≥455 IU/mL in plasma in 2 consecutive assessments within 14 days of the first dose of maribavir, separated by at least 1 day, and where the second sample is obtained within 5 days prior to the first dose of maribavir, by quantitative polymerase chain reaction (qPCR).

27. The method of claim 23, wherein the patient does not have CMV tissue invasive disease involving the central nervous system (CNS) or retina prior to treatment with maribavir.

28. The method of claim 23, further comprising the step of confirming CMV viremia clearance in the patient.

29. (canceled)

30. The method of claim 23, comprising administering maribavir to the patient with or without food.

31. (canceled)

32. The method of claim 23, wherein the patient is refractory to treatment with one or more of ganciclovir, valganciclovir, cidofovir, or foscarnet.

33. (canceled)

34. (canceled)

35. The method of claim 23, wherein the patient is a transplant recipient.

36. The method of claim 23, wherein the patient is a hematopoietic stem cell transplant recipient.

37. The method of claim 23, wherein the patient is a solid organ transplant recipient.

38. The method of claim 23, wherein the patient is less than 6 years of age.

39. The method of claim 23, wherein the patient is receiving or has received an antacid.

40. The method of claim 23, wherein the pharmaceutical composition is administered to a patient who is receiving or has received rabeprazole.

41. The method of claim 23, wherein the patient is administered 50 mg of maribavir once daily.

42. The method of claim 23, wherein the patient is administered 50 mg of maribavir twice daily.

43. The method of claim 23, wherein the patient is administered 100 mg of maribavir twice daily.

44. The method of claim 23, wherein the patient is administered 150 mg of maribavir twice daily.

45. The method of claim 23, wherein the patient is administered 200 mg of maribavir twice daily.

46. The method of claim 23, wherein the patient is administered 300 mg of maribavir twice daily.

47. The method of claim 23, wherein the patient is administered 400 mg of maribavir twice daily.

48. A composition for oral administration of maribavir comprising granules having a median particle diameter of about 225-350 microns, which granules comprise an intragranular composition coated by an enteric coating layer, and wherein the granules are further admixed within extragranular components.

49. The composition of claim 48 comprising granules having a median particle diameter of about 250-325 microns, about 250-310 microns, about 250-300 microns, about 250-290 microns, about 250-280 microns, about 250-270 microns, or about 225-300 microns.

50. A pharmaceutical composition comprising:
(a) maribavir;
(b) a pharmaceutically acceptable carrier or excipient; and
(c) means for masking a perceived bitter flavor of maribavir.

51. The pharmaceutical composition of claim 50, wherein the perceived bitter flavor is reduced by at least 50% compared to maribavir when assessed according to the Flavor Profile Method.

52. (canceled)

53. (canceled)

54. (canceled)

55. The pharmaceutical composition of claim 50, wherein the perceived oral bitterness is reduced by at least 90% compared to maribavir when assessed according to the Flavor Profile Method.

56. A method of preparing a pharmaceutical composition for liquid oral administration, comprising steps of:
i) granulating an active pharmaceutical ingredient and other excipients to form an intragranular composition;
ii) coating the intragranular composition with a selectively soluble polymer to form a coated intragranular composition;
iii) blending the coated intragranular composition, diluent, sweetener, flavorant, and other excipients to form a blended composition;
iv) filling and packaging the blended composition;
wherein median particle diameter of the intragranular composition is between about 100 and 400 microns, wherein median particle diameter of the coated intragranular composition is less than 500 microns, and wherein dv50 particle size of the diluent is between about 30 and 180 microns.

57. (canceled)

58. (canceled)

59. The method of claim 56, further comprising the step of suspending the pharmaceutical composition in a liquid vehicle (e.g., water) prior to oral administration.

60. The method of claim 56, wherein the median particle diameter of the coated intragranular composition is about 225-350 microns.

61. (canceled)

62. (canceled)

63. The method of claim 56, wherein the median particle diameter of the coated intragranular composition is about 250-290 microns.

64. (canceled)

65. The method of claim 56, wherein the median particle diameter of the coated intragranular composition is about 250-270 microns.

66. (canceled)

67. The method of claim 56, wherein the selectively soluble polymer is insoluble at neutral pH but soluble at acidic pH.

68. The method of claim 67, wherein the selectively soluble polymer is Eudragit® E PO.

69. The method of claim 23, wherein the patient is less than 12 years of age.

70. The method of claim 69, wherein the patient is at least 6 years of age, and less than 12 years of age.

71. The pharmaceutical composition of claim 2, wherein the pharmaceutical composition is a powder.
